(19) United States
(12) Patent Application Publication
(10) Pub. No.: US 2021/0346085 A1
(43) Pub. Date: Nov. 11, 2021

(54) ENERGY TREATMENT INSTRUMENT

(71) Applicant: OLYMPUS CORPORATION, Tokyo (JP)

(72) Inventors: Hidenosuke HASE, Hachioji-shi (JP); Satoshi MIYAJIRI, Kokubunji-shi (JP)

(73) Assignee: OLYMPUS CORPORATION, Tokyo (JP)

(21) Appl. No.: 17/380,616

(22) Filed: Jul. 20, 2021

Related U.S. Application Data
(63) Continuation of application No. PCT/JP2019/002133, filed on Jan. 23, 2019.

(51) Int. Cl. A61B 18/14

(52) U.S. Cl. CPC A61B 18/1445; A61B 2018/00607

(57) ABSTRACT

An energy treatment instrument includes: a grip configured to be gripped by an operator; a transducer configured to generate ultrasound vibrations according to a supplied electric power; a first ultrasound terminal that is arranged in the transducer; a first high-frequency terminal that is arranged in the grip; and a base unit that is arranged in the grip. The base unit includes a second ultrasound terminal configured to electrically connect the first ultrasound terminal and a cable electrically connected to a control device outside the energy treatment instrument, by abutting on the first ultrasound terminal; and a second high-frequency terminal configured to electrically connect the first high-frequency terminal and a cable electrically connected to the control device outside the energy treatment instrument, by abutting on the first high-frequency terminal.

### CROSS-REFERENCE TO RELATED APPLICATION

This application is a continuation of International Application No. PCT/JP2019/002133, filed on Jan. 23, 2019, the entire contents of which are incorporated herein by reference.

### BACKGROUND

### 1. Technical Field

The present disclosure relates to an energy treatment instrument.

### 2. Related Art

Energy treatment instruments to treat an area targeted for treatment (hereinafter, “target area”) in a living tissue by applying an energy to the target area have been known (for example, WO2014/007168).

An energy treatment instrument described in WO2014/007168 includes a handle unit, and a transducer unit that is arranged detachably to the handle unit.

The handle unit includes an end effector that applies an ultrasound energy to treat a target area, and a grip that supports the end effector, and that is gripped by an operator.

The transducer unit generates ultrasound vibrations according to a supplied electric power. The transducer unit transmits the generated ultrasound vibrations to the end effector. Thus, the end effector transmits the ultrasound vibrations to the target area. That is, to the target area, an ultrasound energy is applied from the end effector.

### SUMMARY

In some embodiments, an energy treatment instrument includes: a grip configured to be gripped by an operator; a transducer configured to generate ultrasound vibrations according to a supplied electric power; a first ultrasound terminal that is arranged in the transducer; a first high-frequency terminal that is arranged in the grip; and a base unit that is arranged in the grip. The base unit includes a second ultrasound terminal configured to electrically connect the first ultrasound terminal and a cable electrically connected to a control device outside the energy treatment instrument, by abutting on the first ultrasound terminal; and a second high-frequency terminal configured to electrically connect the first high-frequency terminal and a cable electrically connected to the control device outside the energy treatment instrument, by abutting on the first high-frequency terminal.

In some embodiments, an energy treatment instrument includes: an end effector configured to apply an energy to treat a living tissue; a grip configured to support the end effector, the grip being gripped by an operator; a first terminal that is electrically connected to the end effector, and that is arranged rotatably together with the end effector relative to the grip about a center axis of the end effector; a switch device configured to accept an operation by an operator; and a base unit that is arranged in the grip. The base unit includes a second terminal configured to electrically connect the first terminal and a cable electrically connected to a control device outside the energy treatment instrument, by abutting on the first terminal; a circuit board configured to relay between the second terminal and the cable; and a flexible board configured to relay between the switch device and the circuit board.

In some embodiments, an energy treatment instrument includes: a transducer that includes a first terminal, the transducer being configured to generate ultrasound vibrations by an electric power supplied from the first terminal; a grip that is gripped by an operator; and a base unit that is arranged in the grip. The base unit includes a second terminal configured to abut on the first terminal; a cable configured to electrically connect the second terminal and a control device outside the energy treatment instrument; and a base member including a fixing portion that is connected to the second terminal and the cable, the fixing portion being configured to fix the second terminal and the cable in the grip.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating a treatment system according to an embodiment;

FIG. 2 is a diagram for explaining a structure of an energy treatment instrument;

FIG. 3 is a diagram for explaining the structure of the energy treatment instrument;

FIG. 4 is a diagram for explaining the structure of the energy treatment instrument;

FIG. 5 is a diagram for explaining the structure of the energy treatment instrument;

FIG. 6 is a diagram for explaining a structure of the energy treatment instrument;

FIG. 7 is a diagram for explaining a positional relationship between a first and a second pins;

FIG. 8 is a diagram illustrating an entire structure of a base unit;

FIG. 9 is a diagram illustrating an entire structure of a base unit;

FIG. 10 is a diagram illustrating a structure of a second terminal-holding unit;

FIG. 11 is a diagram illustrating the structure of the second terminal-holding unit;

FIG. 12 is a diagram illustrating a circuit board;

FIG. 13 is a circuit diagram for detecting an operation made with respect to a first to a third switches;

FIG. 14 is a diagram for explaining a supporting structure of the third switch;

FIG. 15 is a diagram for explaining the supporting structure of the third switch;

FIG. 16 is a diagram for explaining the supporting structure of the third switch;

FIG. 17 is a diagram for explaining the supporting structure of the third switch;

FIG. 18 is a flowchart of a manufacturing method of the energy treatment instrument;

FIG. 19 is a diagram for explaining the manufacturing method of the energy treatment instrument;

FIG. 20 is a diagram for explaining the manufacturing method of the energy treatment instrument;

FIG. 21 is a diagram for explaining the manufacturing method of the energy treatment instrument;

FIG. 22 is a diagram for explaining the manufacturing method of the energy treatment instrument; and

FIG. 23 is a diagram illustrating a modification of the embodiment.

### DETAILED DESCRIPTION

A form (hereinafter, embodiment) to implement the disclosure will be explained with reference to the accompanying drawings. The embodiment explained below is not intended to limit the disclosure. Furthermore, like reference symbols are assigned to like parts throughout the drawings.

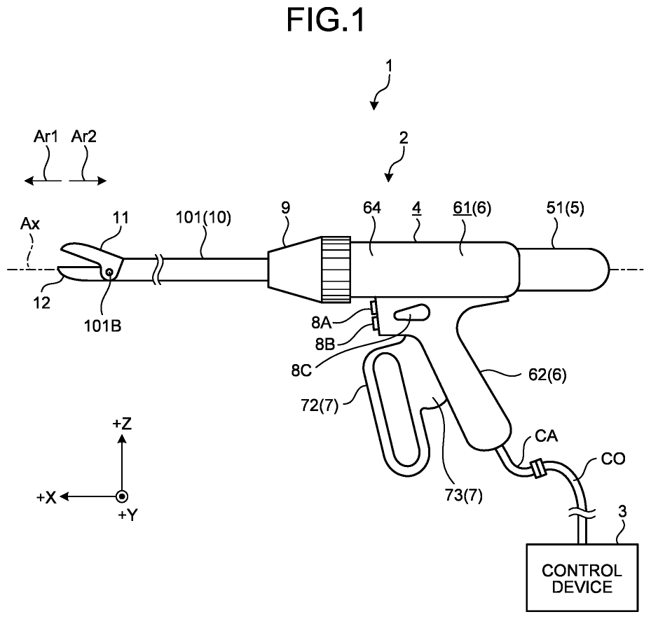

Schematic Structure of Treatment System FIG. 1 is a diagram illustrating a schematic structure of a treatment system 1 according to a present embodiment.

The treatment system 1 applies a ultrasound energy and a high frequency energy to an area targeted for treatment (hereinafter, target area) in a living tissue, to treat the target area. Treatments enabled by the treatment system 1 according to the present embodiment are treatments including coagulation (sealing) of a target area, incision of a target area, or the like. Moreover, it may be a treatment in which coagulation and incision are performed at the same time. The treatment system 1 includes an energy treatment instrument 2 and a control device 3 as illustrated in FIG. 1.

Structure of Energy Treatment Instrument

In the following, to explain a structure of the energy treatment instrument 2, XYZ coordinate axes of an X axis, a Y axis, and a Z axis that are perpendicular to one another are used. The X axis is an axis parallel to a center axis Ax (FIG. 1) of a sheath 10. The Y axis is an axis perpendicular to a sheet plane of FIG. 1. The Z axis is an axis along an upward downward direction of FIG. 1. Furthermore, in the following, one side (+X axis side) along the center axis Ax is denoted as a distal end side Ar1, and the other side (−X axis side) is denoted as a proximal end side Ar2.

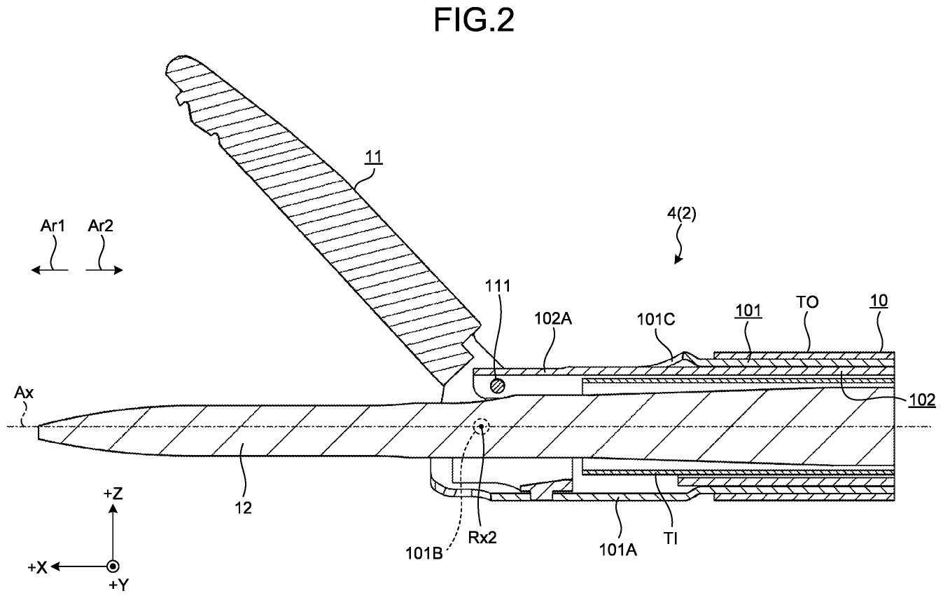

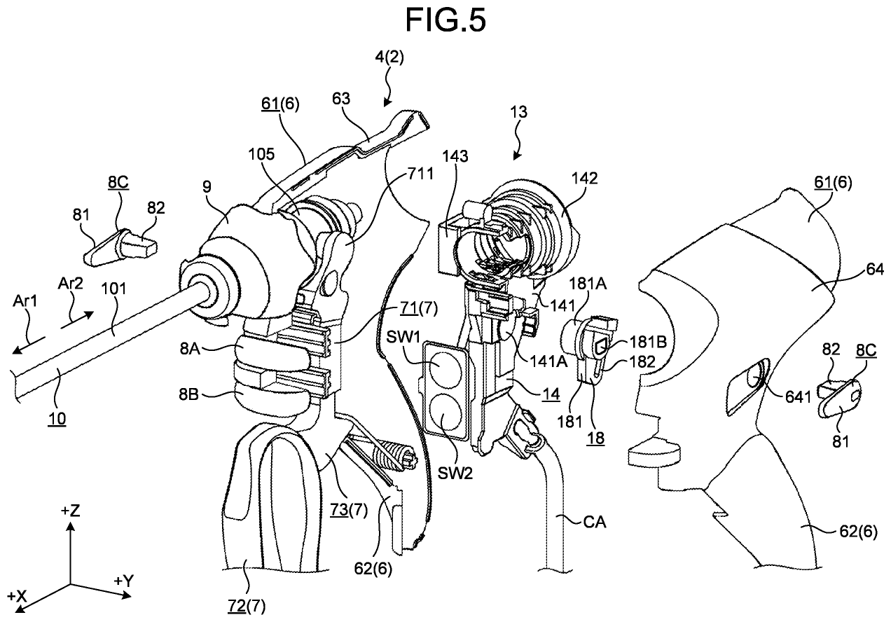

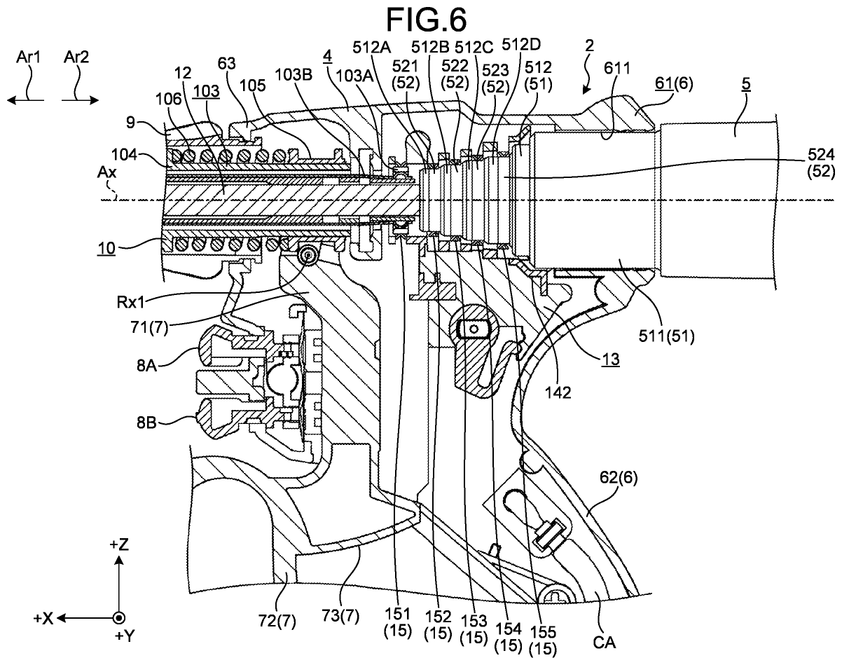

FIG. 2 to FIG. 6 are diagrams for explaining a structure of the energy treatment instrument 2. Specifically, FIG. 2 to FIG. 4 illustrate a part of a cross-section of the energy treatment instrument 2 cut along an XZ plane including the center axis Ax viewed from a +Y axis side, sequentially from the distal end side Ar1 toward the proximal end side Ar2. FIG. 5 and FIG. 6 are diagrams illustrating an inside of a holding case 6. In FIG. 5, illustration of an ultrasound transducer 5 is omitted for convenience of explanation.

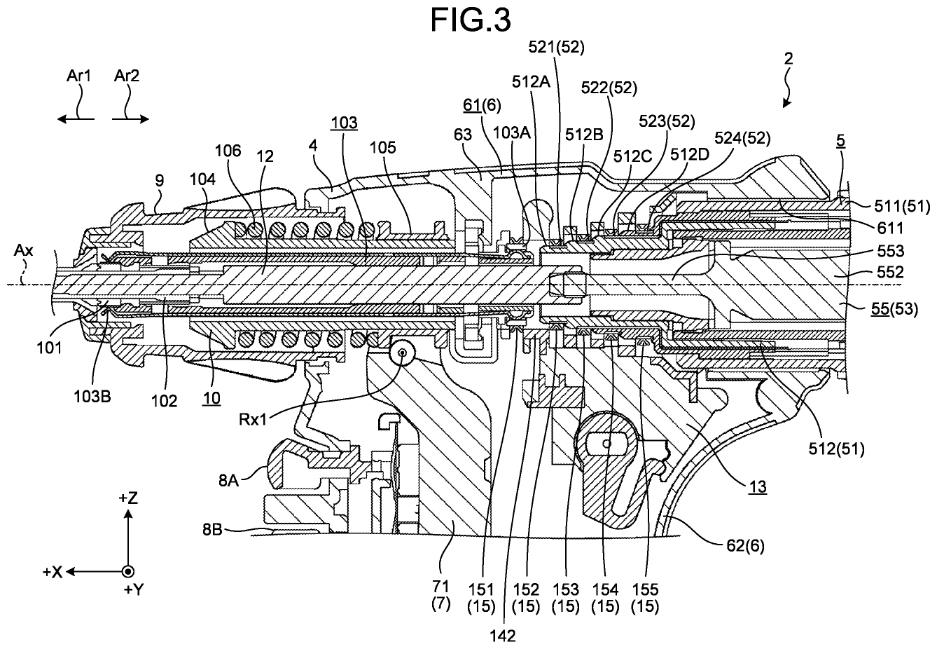

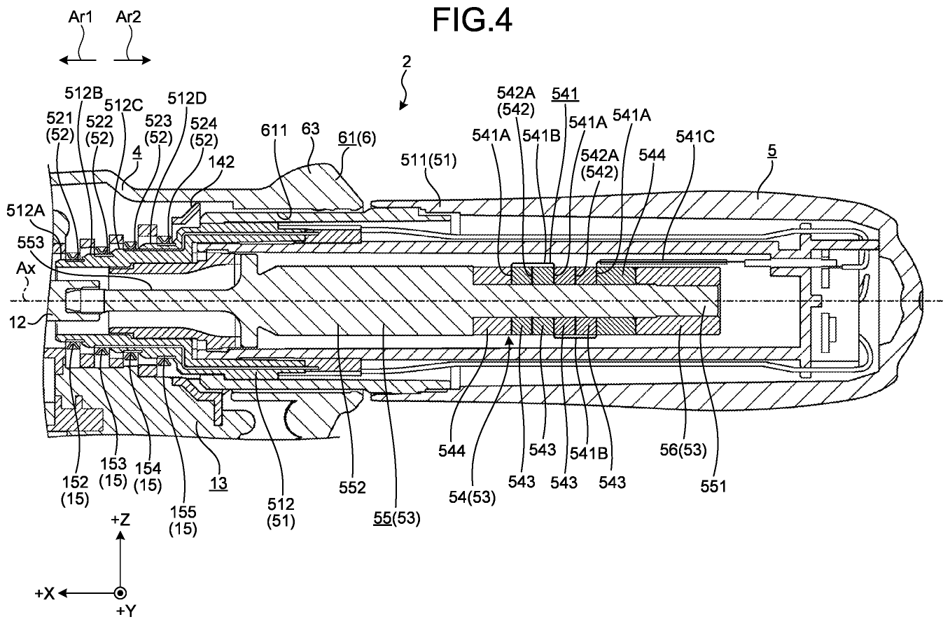

The energy treatment instrument 2 is a surgical treatment instrument to treat a target area, for example, through an abdominal wall. This energy treatment instrument 2 includes a handpiece 4 and the ultrasound transducer 5 (FIG. 1, FIG. 3, FIG. 4, FIG. 6) as illustrated in FIG. 1 to FIG. 6.

The handpiece 4 includes the holding case 6 (FIG. 1, FIG. 3 to FIG. 6), a movable handle 7 (FIG. 1, FIG. 3, FIG. 5, FIG. 6), a first switch 8A (FIG. 1, FIG. 3, FIG. 5, FIG. 6), a second switch 8B (FIG. 1, FIG. 3, FIG. 5, FIG. 6), a pair of third switches 8C (FIG. 1, FIG. 5), a rotating knob 9 (FIG. 1, FIG. 3, FIG. 5, FIG. 6), the sheath 10 (FIG. 1 to FIG. 3, FIG. 5, FIG. 6), a jaw 11 (FIG. 1, FIG. 2), an ultrasound probe 12 (FIG. 1 to FIG. 4, FIG. 6), a base unit 13 (FIG. 3 to FIG. 6), and a cable CA (FIG. 1, FIG. 5, FIG. 6) as illustrated in FIG. 1 to FIG. 6.

The holding case 6 corresponds to a grip. This holding case 6 holds the entire part of the energy treatment instrument 2. The holding case 6 includes, as illustrated in FIG. 5, a holding-case main body 61 in a substantially cylindrical shape coaxial to the center axis Ax, and a fixed handle 62 that extends downward in FIG. 5 from the holding-case main body 61, and that is gripped by an operator such as a surgeon.

In the present embodiment, the holding case 6 is divided into two pieces at the XZ plane including the center axis Ax as a boundary. The holding case 6 is structured by combining these two pieces. In the following, out of the two pieces, a portion on a −Y axis side is denoted as a first housing 63 (FIG. 3 to FIG. 6), and a portion on the +Y axis side is denoted as a second housing (FIG. 5).

The movable handle 7 accepts respective operations of a closing operation and an opening operation by an operator such as a surgeon. This movable handle 7 includes a handle base portion 71, an operating portion 72, and a connecting portion 73 as illustrated in FIG. 5.

The handle base portion 71 is positioned inside the holding case 6. A portion on a +Z axis side in the handle base portion 71 is supported rotatably about a first rotation axis Rx1 (FIG. 3, FIG. 6) parallel to the Y axis with respect to the holding case 6. Moreover, to an end portion of the handle base portion 71 on the +Z axis side, a pair of engaging portions 711 (FIG. 5) that protrude toward the +Z axis side in a branched state into two, and that engage with a slider 105 constituting the sheath 10 are arranged.

The operating portion 72 is a portion that accepts respective operations of a closing operation and an opening operation by an operator such as a surgeon, and is positioned outside the holding case 6 as illustrated in FIG. 5.

The connecting portion 73 is a portion that is arranged, lying across the inside and the outside of the holding case 6 as illustrated in FIG. 5, and is a portion that connects the handle base portion 71 and the operating portion 72.

The movable handle 7 rotates in a counterclockwise direction about the rotation axis Rx1 in FIG. 3 when a closing operation by an operator such as a surgeon is accepted. That is, the operating portion 72 moves toward a direction of approaching the fixed handle 62. On the other hand, when an opening operation with respect to the movable handle 7 is accepted, the movable handle 7 rotates in a clockwise direction about the first rotation axis Rx1 in FIG. 3. That is, the operating portion 72 moves toward a direction of moving apart from the fixed handle 62.

A part of the connecting portion 73 is positioned always inside the holding case 6 throughout states of the movable handle 7 being rotated fully in the counterclockwise direction about the rotation axis Rx1 in FIG. 3 by a closing operation and of the movable handle 7 being fully rotated in the clockwise direction about the rotation axis Rx1 in FIG. 3 by an opening operation. At the closing operation, a direction between the fixed handle 62 and the movable handle 7 decreases. The connecting portion 73 is designed such that there is no portion at which a distance between the fixed handle 62 and the movable handle 7 at this time is smaller than an outer diameter of the cable CA. Thus, the cable CA is prevented from being sandwiched between the fixed handle 62 and the movable handle 7.

The first and the second switches 8A, 8B are respectively positioned at a separation position of the first and the second housings 63, 64 as illustrated in FIG. 5, and are respectively arranged in an exposed state to outside from a side surface of the fixed handle 62 on the distal end side Ar1.

The first switch 8A accepts a setting operation of a first energy output mode by an operator such as a surgeon.

Moreover, the second switch 8B accepts a setting operation of a second energy output mode by an operator such as surgeon. The second energy output mode is an energy output mode different from the first energy output mode.

An example of the first energy output mode is an energy mode to perform coagulation and incision of a target area by applying an ultrasound energy and a high frequency energy. Furthermore, an example of the second energy output mode is an energy output mode to perform coagulation of a target area by applying a high frequency energy.

The pair of third switches 8C oppose to each other along the Y axis as illustrated in FIG. 5, and are arranged in an exposed state to outside from the first and the second housings 63, 64.

The pair of third switches 8C accepts a change operation to change an output state in at least one of the energy output modes of the first and the second energy output modes by an operator such as a surgeon. The change of an output state in the energy output mode is to switch, for example, between a high output mode that drives at a relatively high voltage, and a low output mode that drives at a lower voltage than the high output mode. Alternatively, it may be configured such that the control device 3 can arbitrarily set what is switched by the third switch 8C.

The pair of third switches 8C are axially supported respectively by the base unit 13, and operate together according to a change operation by an operator such as a surgeon. A structure of the third switches 8C and a supporting structure of the base unit to support the third switches 8C will be described later in “Structure of Base Unit”.

The rotating knob 9 has a substantially cylindrical shape coaxial to the center axis Ax, and is arranged at the distal end side Ar1 of the holding-case main body 61 as illustrated in FIG. 5. The rotating knob 9 accepts a rotation operation by an operator such as a surgeon. By the rotation operation, the rotating knob 9 rotates about the center axis Ax relative to the holding-case main body 61. Moreover, with the rotation of the rotating knob 9, the jaw 11 and the ultrasound probe 12 rotate about the center axis Ax.

The sheath 10 has substantially cylindrical shape as a whole. This sheath 10 includes, as illustrated in FIG. 1 to FIG. 3, FIG. 5, or FIG. 6, an outer pipe 101 (FIG. 1 to FIG. 3, FIG. 5), an inner pipe 102 (FIG. 2, FIG. 3), a probe holder 103 (FIG. 3, FIG. 6), a slider receiver 104 (FIG. 3, FIG. 6), and the slider 105 (FIG. 3, FIG. 5, FIG. 6).

The outer pipe 101 is a cylindrical pipe made from a conductive material, such as a metal.

The ultrasound probe 12 vibrates with a large ultrasound energy. Therefore, there is a possibility that the ultrasound probe 12 is damaged if the vibrating ultrasound probe 12 touches the outer pipe 101 made from a metal or the like. Moreover, as described later, because the ultrasound probe 12 and the outer pipe 101 form an electrical pathway through which a high frequency energy flows, it is necessary to avoid these components from touching each other. Therefore, in this outer pipe 101, an expanded pipe portion 101A, a diameter of which is expanded to be larger than other portions is arranged at an end portion on the distal end side Ar1 as illustrated in FIG. 2, to avoid contact with the ultrasound probe 12.

Moreover, in the outer pipe 101, an outer peripheral surface of the portions other than the expanded pipe portion 101A is covered with an electrical insulating outer tube TO (FIG. 2, FIG. 3).

In the present embodiment, a length of the expanded pipe portion 101A (length along the center axis Ax) is set to, for example, about 5 millimeters (mm) to 15 mm. That is, by making the length of the expanded pipe portion 101A as short as possible, it is arranged to decrease an exposed portion of the outer pipe 101, and to avoid the outer pipe 101 from touching the ultrasound probe 12.

Furthermore, to the expanded pipe portion 101A, a first pin 101B (FIG. 1, FIG. 2) that extends to a direction perpendicular to the sheet plane of FIG. 1 and FIG. 2, and that axially supports the jaw 11 in a rotatable manner about a second rotation axis Rx2 (FIG. 2) is fixed.

Moreover, in the expanded pipe portion 101A, a notch 101C that extends toward the proximal end side Ar2 from the distal end is formed on the +Z axis side.

The inner pipe 102 is a pipe in a cylindrical shape having a diameter smaller than that of the outer pipe 101. Furthermore, the inner pipe 102 is inserted in the outer pipe 101 coaxially to the outer pipe 101.

In this inner pipe 102, an arm portion 102A that protrudes toward the distal end side Ar1 is arranged at the end portion on the distal end side Ar1 on the +Z axis side as illustrated in FIG. 2. To this arm portion 102A, a second pin 111 that is arranged in the jaw 11, and that extends in parallel with the second rotation axis Rx2 (the first pin 101B) is inserted.

The probe holder 103 is made from a material having an electrical insulation property, such as resin, and has a substantially cylindrical shape. This probe holder 103 is inserted in the rotating knob 9 and the holding-case main body 61 so as to lie astride the rotating knob 9 and the holding-case main body 61 as illustrated in FIG. 3. The probe holder 103 holds the ultrasound probe 12 inserted therein. Moreover, the probe holder 103 mechanically connects the rotating knob 9 and the outer pipe 101 at the end portion on the distal ends side Ar1. That is, the probe holder 103, the outer pipe 101, the jaw 11, and the ultrasound probe 12 rotate about the center axis Ax together with the rotating knob 9 according to a rotation operation made with respect to the rotating knob 9 by an operator such as surgeon.

In this probe holder 103, an HF active-electrode terminal 103A and an electrical pathway 103B are arranged as illustrated in FIG. 3 or FIG. 6.

The HF active-electrode terminal 103A also has a function as a first terminal. This HF active-electrode terminal 103A is made from a conductive material, and has a ring shape present in an entire circumference in a circumferential direction about the center axis Ax. Moreover, the HF active-electrode terminal 103A is attached to an outer circumferential surface of the probe holder 103 on the proximal end side Ar2. The HF active-electrode terminal 103A is electrically connected to an HF active-electrode terminal 151 (FIG. 3, FIG. 6) arranged in the base unit 13. Because the HF active-electrode terminal 103A has a ring shape as described above, even when rotated about the center axis Ax with respect to the HF active-electrode terminal 151 according to a rotation operation made with respect to the rotating knob 9 by an operator such as surgeon, the HF active-electrode terminal 103A is electrically connected to the HF active-electrode terminal 151 all the time.

The electrical pathway 103B is made from a conductive material, and extends from the end portion on the proximal end side Ar2 to the end portion on the distal end side Ar1 on the outer circumferential surface of the probe holder 103. The electrical pathway 103B is electrically connected to the HF active-electrode terminal 103A at the end portion on the proximal end side Ar2 as illustrated in FIG. 3, and is electrically connected to the outer pipe 101 at the end portion on the distal end side Ar1.

The slider receiver 104 is made from a material having an electrical insulation property, such as resin, and has a substantially cylindrical shape. The slider receiver 104 is arranged movably along the center axis Ax relative to the probe holder 103 in a state in which the probe holder 103 is inserted therein. An end portion of the slider receiver 104 on the distal end side Ar1 is fixed to an end portion of the inner pipe 102 on the proximal end side Ar2 in a state in which rotation about the center axis Ax is controlled while being allowed to move along the center axis Ax relative to the probe holder 103. That is, the slider receiver 104 and the inner pipe 102 rotate about the center axis Ax with the rotating knob 9 according to a rotation operation made with respect to the rotating knob 9 by an operator such as a surgeon.

The slider 105 has a substantially cylindrical shape, and is arranged movably along the center axis Ax relative to the slider receiver 104 in a state in which the slider receiver is inserted therein. The slider 105 is engaged with the movable handle 7 by the pair of engaging portions 711 as described above.

The slider 105, the slider receiver 104, and the inner pipe 102 move as described below according to an operation made with respect to the movable handle 7 by an operator such as a surgeon.

The slider 105 is pushed toward the distal end side Ar1 along the center axis Ax by the pair of engaging portions 711 according to a closing operation made with respect to the movable handle 7 by an operator such as surgeon. Moreover, the slider receiver 104 receives a pressure pushed toward the distal end side Ar1 from the slider 105 through a coil spring 106 (FIG. 3, FIG. 6) arranged between itself and the slider 105. Furthermore, the inner pipe 102 moves toward the distal end side Ar1 along the center axis Ax together with the slider receiver 104. Moreover, the arm portion 102A pushes the second pin 111 toward the distal end side Ax1. The jaw 11 rotates in a counterclockwise direction in FIG. 2 about the second rotation axis Rx2. At this time, the second pin 111 also moves keeping a constant distance about the second rotation axis Rx2, the arm portion 102A moves toward the distal end side Ar1 while changing its shape toward the +Z axis side at which the notch 101C is arranged. That is, the jaw 11 moves toward a direction approaching an end portion (closing direction) of the ultrasound probe 12 on the distal end side Ar1.

Moreover, the jaw 11 rotates in a clockwise direction in FIG. 2 about the second rotation axis Rx2 according to an opening operation made with respect to the movable handle 7 by an operator such as a surgeon. That is, the jaw 11 moves toward a direction of separating apart (opening direction) from the end portion of the ultrasound probe 12 on the distal end side Ar1.

As described above, the jaw 11 opens and closes relative to the end portion of the ultrasound probe 12 on the distal end side Ar1 according to an operation made with respect to the movable handle 7 by an operator such as a surgeon.

In the present embodiment, a length of the arm portion 102A (length along the center axis Ax) is set to, for example, 5 mm to 10 mm. That is, by making the length of the arm portion 102A as short as possible, contact of the arm portion 102A and the outer pipe 101 at the time when the arm portion 102A changes its shape with opening and closing of the jaw 11 is avoided. Moreover, by forming the arm portion 102A to have a cross-sectional shape perpendicular to the center axis Ax in a substantially U-shape, or in a wide shape, the rigidity of the arm portion 102A is obtained, and contact of the arm portion 102A and the outer pipe 101 at the time when the arm portion 102A changes its shape with opening and closing of the jaw 11 is avoided. With these arrangement, it is possible to prevent reduction of an opening and closing force of the jaw 11 (grip force of a target area) caused by the contact.

Moreover, in the present embodiment, a separation distance between an outer surface of the arm portion 102A and the center axis Ax is set to be equal to or smaller than a separation distance between an outer peripheral surface of portions other than the arm portion 102A in the inner pipe 102 and the center axis Ax. This avoids the arm portion 102A from rubbing against an inner surface of the outer pipe 101 at the time when the inner pipe 102 is inserted into the outer pipe 101 from the proximal end. That is, it is possible to improve assemblability of the inner pipe 102 with respect to the outer pipe 101.

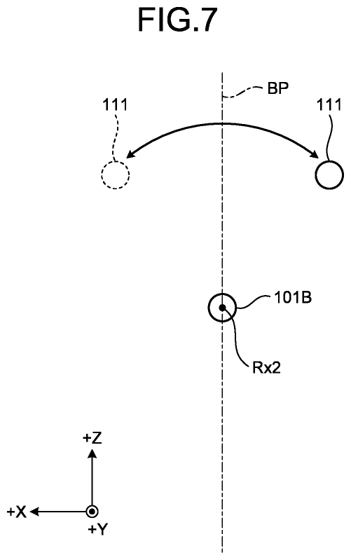

FIG. 7 is a diagram for explaining a positional relationship between the first and the second pins 101B, 111. Specifically, FIG. 7 illustrates the first and the second pins 101B, 111 viewed along a direction perpendicular to the sheet plane of FIG. 2. In FIG. 7, the second pin 111 in a state in which the jaw 11 is open is illustrated by a solid line, and the second pin 111 in a state in which the jaw 11 is closed is illustrated by a broken line.

In the present embodiment, as illustrated in FIG. 7, it is arranged such that a YZ plane BP (FIG. 7) passing through the second rotation axis Rx2 is positioned between a position of the second pin 111 in the state in which the jaw 11 is open, and a position of the second pin 111 in the state in which the jaw 11 is closed. It is more preferable to structure such that the second pin 111 in the state in which the jaw 11 is open and the second pin 111 in the state in which the jaw 11 is closed are arranged at symmetrical positions relative to the plane BP. This arrangement enables to minimize a deformation of the arm portion 102A in the Z axis direction with opening and closing of the jaw 11, and to convert a force occurring movement of the inner pipe 102 along the center axis Ax into a force to open and close the jaw 11 (force to grip a target area).

At least a part of the jaw 11 is made from a conductive material. The jaw 11 is electrically connected to the HF active-electrode terminal 103A through the outer pipe 101 and the electrical pathway 103B.

The ultrasound probe 12 corresponds to an end effector. This ultrasound probe 12 is made from a conductive material, and has a long shape linearly extending along the center axis Ax. Moreover, the ultrasound probe 12 is inserted in the sheath 10 in a state in which the end portion on the distal end side Ar1 protrudes outside as illustrated in FIG. 2. In this state, an end portion of the ultrasound probe 12 on the proximal end side Ar2 is mechanically connected to the ultrasound transducer 5 as illustrated in FIG. 3 or FIG. 6. That is, the ultrasound transducer 5 rotates about the center axis Ax together with the ultrasound probe 12 according to a rotation operation made with respect to the rotating knob 9 by an operator such as a surgeon. The ultrasound probe 12 transmits ultrasound vibrations generated by the ultrasound transducer 5 from the end portion on the proximal end side Ar2 to the end portion on the distal end side Ar1. In the present embodiment, the ultrasound vibrations are axial vibrations vibrating in the direction along the center axis Ax.

An outer surface of the ultrasound probe 12 is covered with an electrically insulating inner tube TI (FIG. 2) to obtain electrical insulation of the ultrasound probe 12 with the outer pipe 101 and the inner pipe 102.

The cable CA is detachably connected to an electric cable CO (FIG. 1) extending from the control device 3. That is, the cable CA is electrically connected to the control device 3 through the electric cable CO.

To the base unit 13, the cable CA is attached as illustrated in FIG. 3 to FIG. 6, and the base unit 13 is arranged inside the holding case 6. This base unit 13 has a function of electrically connecting the cable CA, the HF active-electrode terminal 103A arranged in the probe holder 103, a first terminal 52 (FIG. 4, FIG. 6) arranged in the ultrasound transducer 5, and a function of supporting the pair of third switches 8C.

Detailed structures of the cable CA and the base unit 13 are explained in “Structure of Base Unit” described later.

The ultrasound transducer 5 includes a transducer (TD) case 51, the first terminal 52, and an ultrasound vibrator 53 as illustrated in FIG. 4.

The TD case 51 supports the first terminal 52 and the ultrasound vibrator 53, and is detachably connected to the holding-case main body 61. This TD case 51 includes a TD-case main body 511 and a first terminal-holding portion 512 as illustrated in FIG. 4.

The TD-case main body 511 has a bottomed cylindrical shape as illustrated in FIG. 4, and is connected to the holding-case main body 61 in a state in which an opening portion is directed to the distal end side Ar1.

On an inner surface of the holding-case main body 61, a guiding surface 611 (FIG. 4, FIG. 6) that extends linearly along the center axis Ax from the proximal end side Ar2 toward the distal end side Ar1, and that has an inner diameter a little larger than an outer diameter of the TD-case main body 511 is arranged at a portion on the proximal end side Ar2. Thus, the ultrasound transducer 5 is guided by the guiding surface 611 on an outer peripheral surface of the TD-case main body 511 when the ultrasound transducer 5 is inserted (connected) to the holding-case main body 61. A center axis of the ultrasound transducer 5 coincides with the center axis Ax. Moreover, it is possible to avoid the ultrasound transducer 5 from hitting against the second terminal-holding portion 142 (FIG. 6) arranged in the base unit 13 when the ultrasound transducer 5 is inserted into the holding-case main body 61 from any angle.

The first terminal-holding portion 512 is a cylindrical body that extends along the center axis Ax as illustrated in FIG. 4, and is engaged in the opening portion of the TD-case main body 511. In this first terminal-holding portion 512, an outer surface of a portion protruding toward the distal end side Ar1 from the TD-case main body 511 is formed in a stepped form having four steps 512A to 512D sequentially from the distal end side Ar1 as illustrated in FIG. 3, FIG. 4, or FIG. 6. These four steps 512A to 512D respectively have a circular cross-sectional shape having the center axis Ax as its center, and diameter sizes thereof increase sequentially from the four steps 512A to 512D.

The first terminal 52 includes an HF return-electrode terminal 521, an IR terminal 522, a US return-electrode terminal 523, and a US active-electrode terminal 524 as illustrated in FIG. 3, FIG. 4, or FIG. 6. The respective terminals 521 to 524 are respectively made from a conductive material.

The HF return-electrode terminal 521 corresponds to a high frequency terminal. This HF return-electrode terminal 521 is arranged on the step 512A throughout an entire periphery in a peripheral direction of a cross-sectional circular shape in the step 512A. The HF return-electrode terminal 521 is electrically connected to an HF return-electrode terminal 152 (FIG. 3, FIG. 4, FIG. 6) arranged in the base unit 13 by connecting the ultrasound transducer 5 to the holding-case main body 61. Because the HF return-electrode terminal 521 is arranged throughout the entire periphery in a peripheral direction of the cross-sectional circular shape in the step 512A as described above, even when rotated about the center axis Ax relative to the HF return-electrode terminal 152 according to a rotation operation made with respect to the rotating knob 9 by an operator such as a surgeon, the HF return-electrode terminal 521 is electrically connected to the HF return-electrode terminal 152 all the time.

The IR terminal 522 is arranged on the step 512B throughout an entire periphery in a peripheral direction of a cross-sectional circular shape in the step 512B. The IR terminal 522 is electrically connected to an IR terminal 153 (FIG. 3, FIG. 4, FIG. 6) arranged in the base unit 13 by connecting the ultrasound transducer 5 to the holding-case main body 61. Because the IR terminal 522 is arranged throughout the entire periphery in the peripheral direction of the cross-sectional circular shape in the step 512B as described above, even when rotated about the center axis Ax relative to the IR terminal 153 according to a rotation operation made with respect to the rotating knob 9 by an operator such as a surgeon, the IR terminal 522 is electrically connected to the IR terminal 153 all the time. Moreover, although specific illustration is omitted, to the ultrasound transducer 5, for example, a transducer (TD) memory storing identification information to identify the ultrasound transducer 5 is included therein. The IR terminal 522 is electrically connected to the TD memory through an electrical pathway (not illustrated) arranged inside the TD case 51.

The US return-electrode terminal 523 corresponds to a first ultrasound terminal. This US return-electrode terminal 523 is arranged on the step 512C throughout an entire periphery in a peripheral direction of a cross-sectional circular shape in the step 512C. The US return-electrode terminal 523 is electrically connected to a US return-electrode terminal 154 (FIG. 3, FIG. 4, FIG. 6) described later by connecting the ultrasound transducer 5 to the holding-case main body 61. Because the US return-electrode terminal 523 is arranged throughout the entire periphery in the peripheral direction of the cross-sectional circular shape in the step 512C as described above, even when rotated about the center axis Ax relative to the US return-electrode terminal 154 according to a rotation operation made with respect to the rotating knob 9 by an operator such as a surgeon, the US return-electrode terminal 523 is electrically connected to the US return-electrode terminal 154 all the time.

The US active-electrode terminal 524 corresponds to the first ultrasound terminal. It is arranged on the step 512D throughout an entire periphery in a peripheral direction of a cross-sectional circular shape in the step 512D. The US active-electrode terminal 524 is electrically connected to a US active-electrode terminal 155 (FIG. 3, FIG. 4, FIG. 6) arranged in the base unit 13 by connecting the ultrasound transducer 5 to the holding-case main body 61. Because the US active-electrode terminal 524 is arranged throughout the entire periphery in the peripheral direction of the cross-sectional circular shape in the step 512D as described above, even when rotated about the center axis Ax with relative to the US active-electrode terminal 155 according to a rotation operation made with respect to the rotating knob 9 by an operator such as a surgeon, the US active-electrode terminal 524 is electrically connected to the US active-electrode terminal 155 all the time.

The ultrasound vibrator 53 generate ultrasound vibrations under control of the control device 3. In the present embodiment, the ultrasound vibrator 53 is constituted of a bolt-clamped Langevin type transducer (BLT). This ultrasound vibrator 53 includes a vibrator main body 54, a front mass 55, and a back mass 56 as illustrated in FIG. 4.

The vibrator main body 54 includes a first and a second electrode plates 541, 542, and plural (four pieces in the present embodiment) piezoelectric devices 543 as illustrated in FIG. 4.

The first and the second electrode plates 541, 542 are portions to which a driving signal being an alternating current power to generate ultrasound vibrations is supplied from the control device 3.

The first electrode plate 541 includes plural (three pieces in the present embodiment) negative electrode plates 541A, plural (two pieces in the present embodiment) negative-electrode wiring portions 541B, and a negative electrode terminal 541C as illustrated in FIG. 4.

The negative electrode plates 541A respectively have a disc shape having an opening in the center (not illustrated), and are arranged to be aligned along the center axis Ax.

The negative-electrode wiring portions 541B are portions to connect outer edge portions of the negative electrode plates 541A adjacent to each other.

The negative electrode terminal 541C extends from an outer edge of the negative electrode plate 541A that is positioned at the most proximal end side Ar2 out of the plural negative electrode plates 541A toward the proximal end side Ar2. The negative electrode terminal 541C is electrically connected to the US return-electrode terminal 523 through the electrical pathway (not illustrated) arranged in the TD case 51. That is, the first electrode plate 541 is electrically connected to the US return-electrode terminal 523.

The second electrode plate 542 includes plural (two pieces in the present embodiment) positive electrode plates 542A, a positive-electrode wiring portion (not illustrated), and a positive electrode terminal (not illustrated) as illustrated in FIG. 4.

The positive electrode plates 542A respectively have a disc shape having an opening (not illustrated) in the center, and are arranged to be aligned along the center axis Ax. The positive electrode plate 541A has a substantially the same shape as the negative electrode plate 541A.

The negative electrode plate 541A and the positive electrode plate 542A are alternately arranged along the center axis Ax as illustrated in FIG. 4. The negative electrode plate 541A that is positioned on the most proximal end side Ar2 out of the plural negative electrode plate 541A is arranged to be positioned closer than the positive electrode plate 542A that is positioned on the most proximal end side Ar2 out of the plural positive electrode plates 542A, to the back mass 56.

The positive-electrode wiring portion (not illustrated) is a portion electrically connecting outer edge portions of the positive electrode plates 542A adjacent to each other.

The positive electrode terminal (not illustrated) extends toward the proximal end side Ar2 from an outer edge of the positive electrode plate 542A positioned on the most proximal end side Ar2 out of the plural positive electrode plates 542A. The positive electrode terminal (not illustrated) is electrically connected to the US active-electrode terminal 524 through an electrical pathway (not illustrated) arranged in the TD case 51. That is, the second electrode plate 542 is electrically connected to the US active-electrode terminal 524.

The plural piezoelectric devices 543 respectively have a disc shape having an opening (not illustrated) in the center, and are arranged respectively between the negative electrode plate 541A and the positive electrode plate 542A. That is, the plural piezoelectric devices 543 are laminated along the center axis Ax. The piezoelectric devices 543 repeat expansion and contraction along a lamination direction as a potential difference occurs in the lamination direction along the center axis Ax according to a driving signal provided by the first and the second electrode plates 541, 542. Thus, the ultrasound vibrator 53 generates axial vibrations having a vibration direction in the lamination direction.

The front mass 55 is made from a conductive material, and has a long shape linearly extending along the center axis Ax. This front mass 55 includes a device mounting portion 551, a section-area changing unit 552, and a probe mounting unit 553 as illustrated in FIG. 4.

The device mounting portion 551 is a bolt that linearly extends in the center axis Ax, and is inserted in the respective openings (not illustrated) of the negative electrode plates 541A, the respective openings (not illustrated) of the positive electrode plates 542A, and the respective openings (not illustrated) of the piezoelectric devices 543, respectively. Furthermore, at an end portion on the proximal end side Ar2 in the device mounting portion 551, the back mass 56 that is a nut made from a conductive material is attached.

The section-area changing unit 552 is arranged at an end portion on the distal end side Ar1 in the device mounting portion 551, and is a portion amplifying an amplitude of ultrasound vibrations. Moreover, the section-area changing unit 552 has a circular truncated conical shape in which a diameter size at an end portion on the proximal end side Ar2 is larger than the device mounting portion 551, and a cross-sectional area gradually decreases as an end portion on the distal end side Ar1 extends toward the distal end side Ar1. That is, the negative electrode plates 541A, the positive electrode plates 542A, and the piezoelectric devices 543 are fastened into one piece having a substantially cylindrical shape, by being sandwiched between the section-area changing unit 552 and the back mass 56 in a state in which the device mounting portion 551 is inserted through along the center axis Ax. In the present embodiment, an insulation plate 544 (FIG. 4) is arranged between the section-area changing unit 552 and the negative electrode plate 541A that is positioned on the most distal end side Ar1 out of the plural negative electrode plates 541A, and between the back mass 56 and the negative electrode plate 541 that is positioned on the most proximal end side Ar2 out of the plural negative electrode plates 541A.

The probe mounting unit 553 is arranged at an end portion of the section-area changing unit 552 on the distal end side Ar1, and extends linearly along the center axis Ax as illustrated in FIG. 4. An end portion of the probe mounting unit 553 on the distal end portion Ar1 is mechanically and electrically connected to an end portion of the ultrasound probe 12 on the proximal end side Ar2 by connecting the ultrasound transducer 5 to the holding-case main body 61.

The back mass 56 is electrically connected to the HF return-electrode terminal 521 through an electrical pathway (not illustrated) arranged in the TD case 51. That is, the ultrasound probe is electrically connected to the HF return-electrode terminal 521 through the back mass 56 and the front mass 55. The HF return-electrode terminal 521 is electrically connected also to a TD memory (not illustrated) mounted in the ultrasound transducer 5 through an electrical pathway (not illustrated) arranged in the TD case 51.

Structure of Control Device

To the control device 3, the energy treatment instrument 2 is detachably connected by the electric cable CO. The control device 3 controls an operation of the energy treatment instrument 2 in a centralized manner through the electric cable CO.

Specifically, the control device 3 is electrically connected to the TD memory mounted in the ultrasound transducer 5 through the HF return-electrode terminal 521, the IR terminal 522. the base unit 13, the cable CA, and the electric cable CO. The control device 3 acquires identification information to identify, for example, the ultrasound transducer 5 stored in the TD memory.

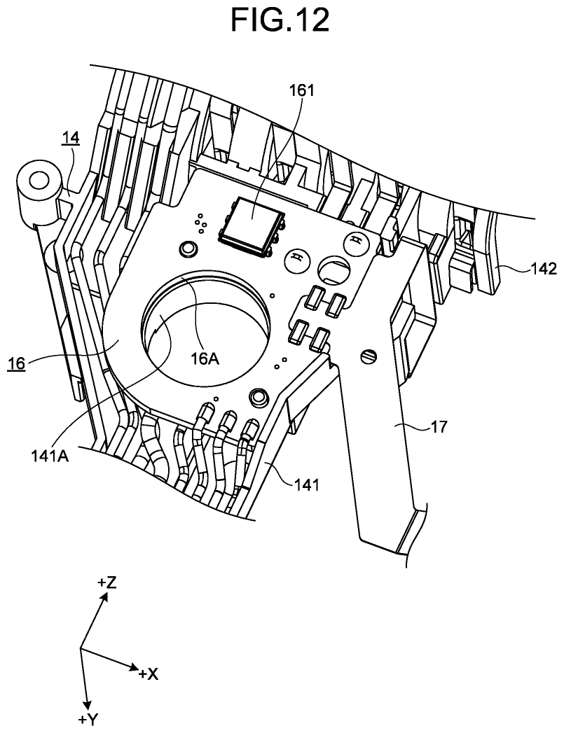

Moreover, the control device 3 is electrically connected to a handpiece memory 161 (refer to FIG. 12) arranged in the base unit 13 through the base unit 13, the cable CA, and the electric cable CO. The control device 3 acquires identification information to identify, for example, the handpiece 4 stored in the handpiece memory 161.

Furthermore, the control device 3 is electrically connected to a first switch device SW1 (FIG. 5) that is arranged in the base unit 13, and that detects a setting operation of the first energy output mode through the base unit 13, the cable CA, and the electric cable CO. That is, the control device 3 is capable of recognizing whether a setting operation of the first energy output mode has been made with respect to the first switch 8A. Moreover, the control device 3 is electrically connected to the first electrode plate 541 through the US return-electrode terminal 523, the base unit 13, the cable CA, and the electric cable CO, and is electrically connected to the second electrode plate 542 through the US active-electrode terminal 524, the base unit 13, the cable CA, and the electric cable CO. Furthermore, the control device 3 is electrically connected to the jaw 11 through the outer pipe 101, the electrical pathway 103B, the HF active-electrode terminal 103A, the base unit 13, the cable CA, and the electric cable CO, and is electrically connected to the ultrasound probe 12 through the front mass 55, the back mass 56, the HF return-electrode terminal 521, the base unit 13, the cable CA, and the electric cable CO.

When a setting operation of the first energy output mode is made with respect to the first switch 8A, the control device 3 performs the first energy output mode as described below.

A case in which an output using a ultrasound energy and a high frequency energy is performed as the first energy output mode will be explained herein. That is, the control device 3 provides a driving signal to the US return-electrode terminal 523 (the first electrode plate 541) and the US active-electrode terminal 524 (the second electrode plate 542). Thus, the piezoelectric devices 543 generates axial vibrations (ultrasound vibrations) vibrating in a direction along the center axis Ax. Moreover, the end portion of the ultrasound probe 12 on the distal end side Ar1 vibrates at a desired amplitude by the axial vibrations. To a target area gripped between the jaw 11 and the end portion of the ultrasound probe 12 on the distal end side Ar1, ultrasound vibrations are applied to the end portion. In other words, the ultrasound energy will be applied to the target area from the end portion.

Moreover, the control device 3 provides a high frequency signal that is a high frequency power to the HF active-electrode terminal 103A (the jaw 11) and the HF return-electrode terminal 521 (the ultrasound probe 12) at substantially the same time as the application of the ultrasound energy to the target area. Thus, the high frequency electric current flows through the target area that is gripped between the jaw 11 and the end portion of the ultrasound probe 12 on the distal end side Ar1. In other words, a high frequency energy is applied to the target area.

In the target area, friction heat is generated between the end portion and the target area by the axial vibrations of the end portion of the ultrasound probe 12 on the distal end side Ar1. Moreover, in the target area, Joule heat is generated as a high frequency electric current flows therethrough. Thus, coagulation (sealing) and incision of the target area are performed.

Moreover, the control device 3 is electrically connected to a second switch device SW2 (FIG. 5) that is arranged in the base unit 13, and that detects a setting operation of the second energy output mode made with respect to the second switch 8B, through the base unit 13, the cable CA, and the electric cable CO. That is, the control device 3 is capable of recognizing whether a setting operation of the second energy output mode is made with respect to the second switch 8B.

The control device 3 performs the second energy output mode as described below when a setting operation of the second energy output mode is made with respect to the second switch 8B.

A case in which an output using a high frequency energy is performed as the second energy output mode will be explained herein. That is, the control device 3 provides a high frequency signal that is a high frequency power to the HF active-electrode terminal 103A (the jaw 11) and the HF return-electrode terminal 521 (the ultrasound probe 12). Thus, the high frequency electric current flows through the target area that is gripped between the jaw 11 and the end portion of the ultrasound probe 12 on the distal end side Ar1.

In the target area, Joule heat is generated as a high frequency electric current flows therethrough. Thus, sealing of the target area is performed.

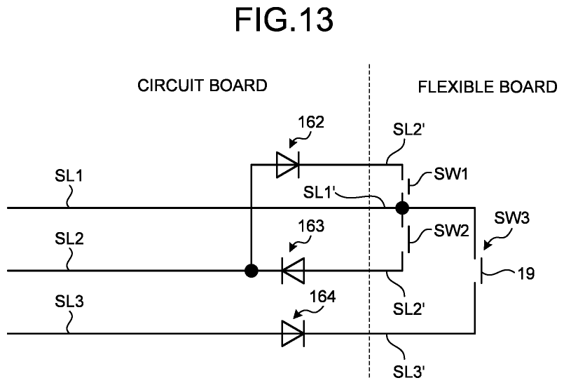

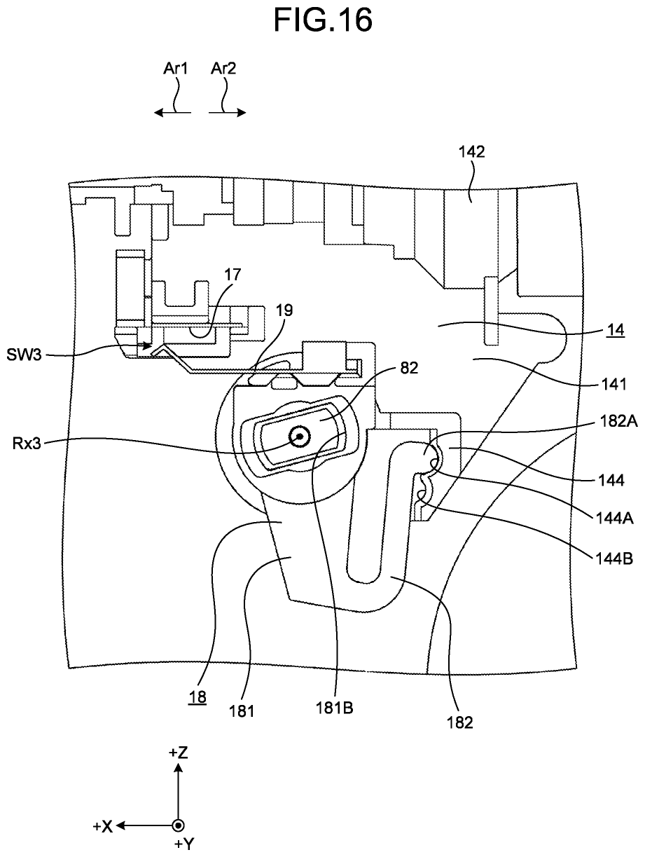

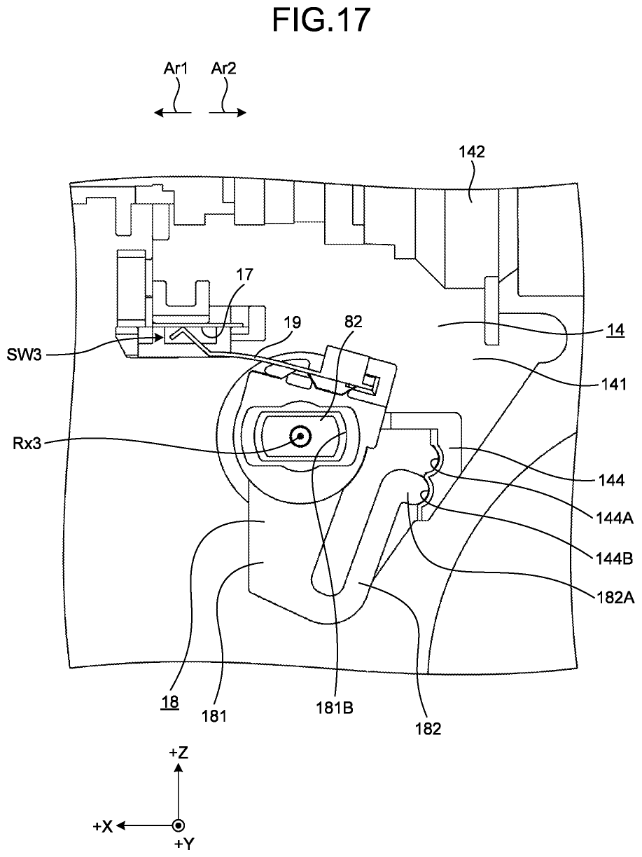

Furthermore, the control device 3 is electrically connected through the base unit 13, the cable CA, and the electric cable CO to a third switch device SW3 (refer to FIG. 13, FIG. 16, FIG. 17) that is arranged in the base unit 13, and that detects a change operation made with respect to the third switch 8C. That is, the control device 3 is capable of recognizing whether a change operation is made with respect to the third switch 8C.

When a change operation is made with respect to the third switch 8C, the control device 3 switches an output state in at least one of the first and the second energy output modes by changing an electric power of the driving signal or the high frequency signal.

Structure of Base Unit Next, a structure of the base unit 13 will be explained.

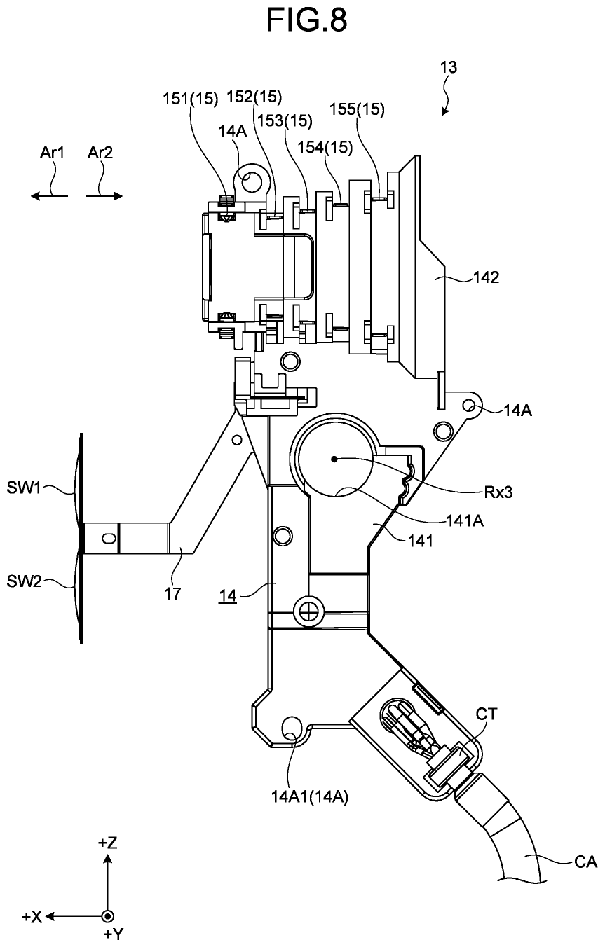

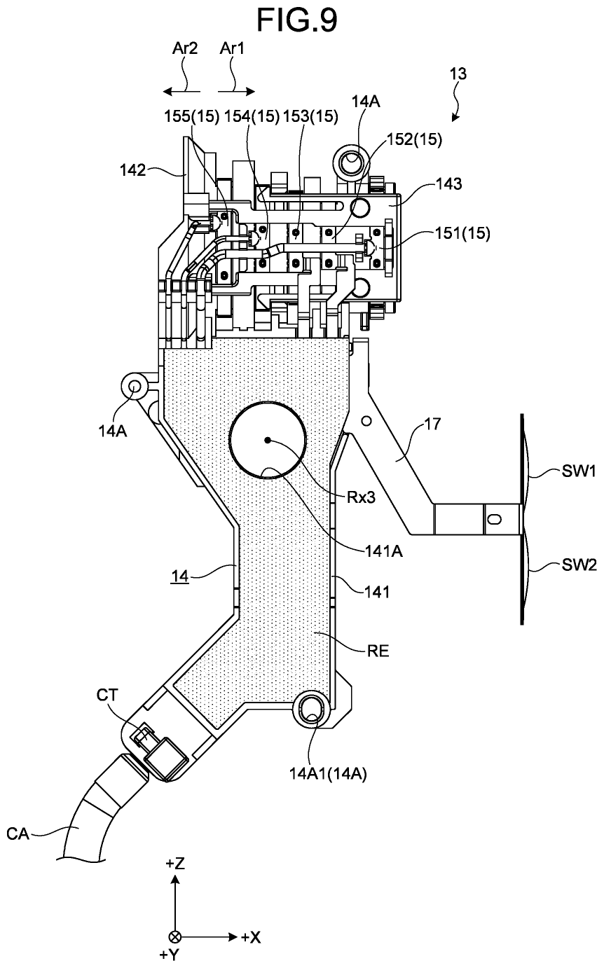

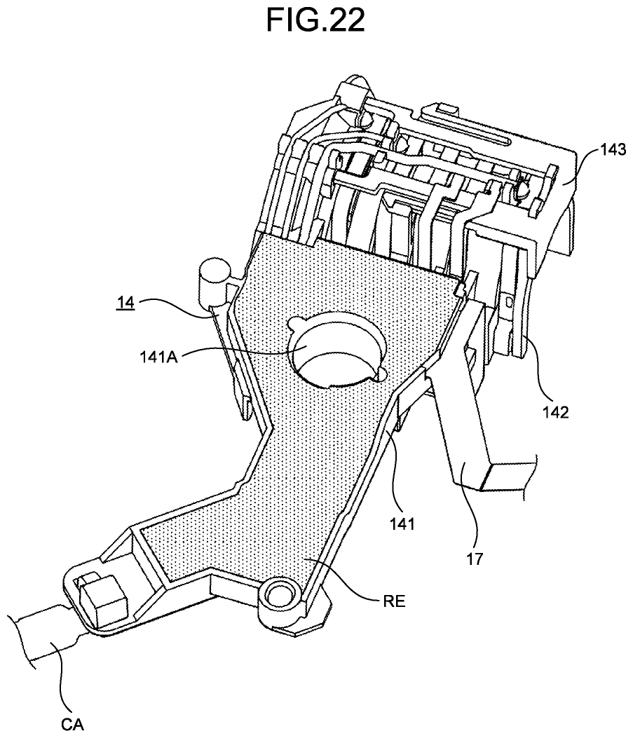

FIG. 8 and FIG. 9 are diagrams illustrating an entire structure of the base unit 13. Specifically, FIG. 8 is a diagram illustrating the base unit 13 viewed from the +Y axis side. FIG. 9 is a diagram illustrating the base unit 13 viewed from the −Y axis side. For convenience of explanation, illustration of a switch supporting portion 18 and a metal contact 19 is omitted in FIG. 8. Moreover, for convenience of explanation, a resin RE is expressed by dots in FIG. 9. The same applies to FIG. 22.

The base unit 13 includes a base member 14, a second terminal 15, a circuit board 16 (refer to FIG. 12), a flexible board 17, the switch supporting portion 18 (FIG. 5), and the metal contact (refer to FIG. 16) attached to the switch supporting portion 18 as illustrated in FIG. 8 or FIG. 9.

The base member 14 is made from a material having an electrical insulation property, and is fixed inside the holding case 6 by plural fixing members 14A (FIG. 8, FIG. 9), such as boss holes. The base member 14 includes a base-member main body 141, a second terminal-holding portion 142, and a terminal pressing member 143 (FIG. 9) as illustrated in FIG. 8 or FIG. 9.

The base-member main body 141 is formed in a plate shape, as illustrated in FIG. 5, and is arranged inside the holding case 6 in such an orientation that respective plate surfaces are parallel to the XZ plane. Moreover, the base-member main body 141 extends from an end portion of the fixed handle 62 on the −Z axis side to the holding-case main body 61 inside the holding case 6.

In the base-member main body 141, a portion at one end of the cable CA is attached to the end portion on the −Z axis side by a tie band CT as illustrated in FIG. 8 or FIG. 9. A portion of the cable CA on the other side is pulled out to an outside of the fixed handle 62 from a side surface of the fixed handle 62 on the −Z axis side. A part of the fixing members 14A1 of the plural fixing members 14A is arranged close to an attachment position of the portion on one end of the cable CA as illustrated in FIG. 8 or FIG. 9. Thus, a load on the base unit 13 caused when the portion on the other end of the cable CA is pulled is reduced. The cable CA may be structured to be detachable to the base unit 13 with a connector.

Moreover, in the base-member main body 141, a circular-shaped bearing hole 141A that pierces through to a front surface to a rear surface, and that axially supports the switch supporting portion 18 in a rotatable manner about the third rotation axis Rx3 parallel to the Y axis is formed at a portion on the +Z axis side as illustrated in FIG. 8 or FIG. 9.

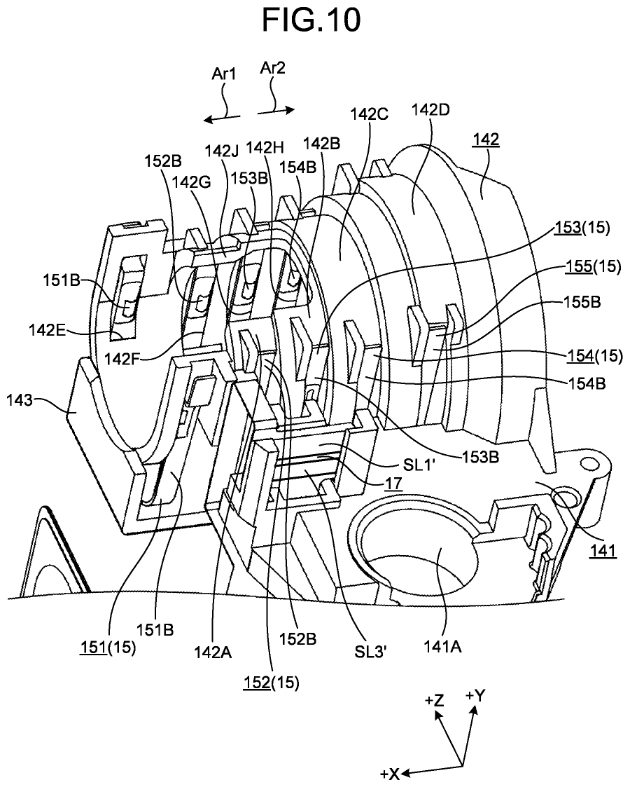

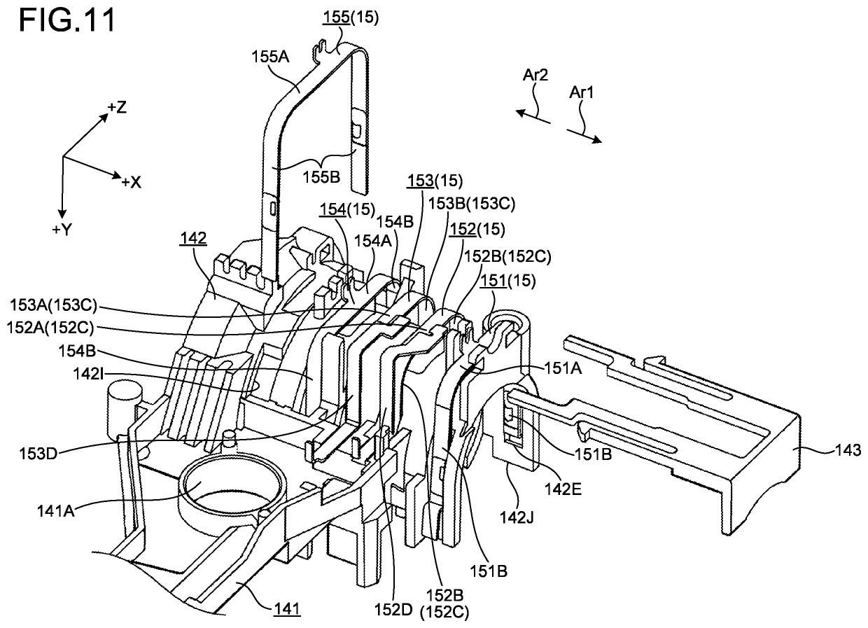

FIG. 10 and FIG. 11 are diagrams illustrating a structure of the second terminal-holding portion 142. Specifically, FIG. 10 is a perspective view of the second terminal-holding portion 142 viewed from the +Y axis side. FIG. 11 is a perspective exploded view of the second terminal-holding portion 142 and the terminal pressing member 143 viewed from the −Y axis side.

The second terminal-holding portion 142 is a tubular member that extends along the X axis (the center axis Ax) as illustrated in FIG. 10 or FIG. 11, and is integrated with the end portion of the base-member main body 141 on the +Z axis side. When the ultrasound transducer 5 is connected to the holding-case main body 61, the first terminal-holding portion 512 in the ultrasound transducer 5 is inserted in the second terminal-holding portion 142 as illustrated in FIG. 3, FIG. 4, or FIG. 6.

An outer surface of this second terminal-holding portion 142 is formed in a stepped form having four steps 142A to 142D sequentially from the distal end side Ar1 as illustrated in FIG. 10. These four steps 142A to 142D respectively have a circular cross-sectional shape having the center axis Ax as its center, and diameter sizes thereof increase sequentially from the four steps 142A to 142D. Moreover, inner diameter sizes of these four steps 142A to 142D are set to be a little larger than the outer diameters of the four steps 512A to 512D in the ultrasound transducer 5. Furthermore, in these four steps 142A to 142D, a pair of openings 142E to 142I that pierces therethrough along the Z axis are formed, respectively, as illustrated in FIG. 10 or FIG. 11.

Moreover, in the second terminal-holding portion 142, a notch 142J that is a portion cut off from the end portion on the distal end side Ar1 t toward the proximal end side Ar2 to a boundary between the steps 142B and 142C is formed on a side surface on the Y axis side as illustrated in FIG. 10 or FIG. 11.

The terminal pressing member 143 is attached on an outer surface of the second terminal-holding portion 142 on the −Y axis side as illustrated in FIG. 11, and is a member that presses the second terminal 15 attached respectively to the four steps 142A to 142D. In the present embodiment, a snap-fit is adopted as a fixing structure of the terminal pressing member 143 to the second terminal-holding portion 142.

The second terminal 15 includes an HF active-electrode terminal 151, an HF return-electrode terminal 152, an IR terminal 153, a US return-electrode terminal 154, and a US active-electrode terminal 155 as illustrated in FIG. 10 or FIG. 11. These respective terminals 151 to 155 are made from a conductive material.

The US active-electrode terminal 155 corresponds to a second and a fourth ultrasound terminals. This US active-electrode terminal 155 includes a terminal base portion 155A and a pair of blade spring portions 155B as illustrated in FIG. 11, and has a substantially U-shape as a whole.

The terminal base portion 155A has a plate shape extending along the Z axis, and is a portion fixed to the outer surface on the −Y axis side in the step 142D in such an orientation that respective plate surfaces are perpendicular to the Y axis.

The pair of blade spring portions 155B are portions respectively extending toward the +Y axis direction from both ends of the terminal base portion 155A, and are formed be elastic to be deformable respectively toward the Z axis direction pivoted on the respective ends. Moreover, in a state in which the terminal base portion 155A is fixed to the step 142D, a part of each of the pair of blade spring portions 155B exposes inside the second terminal-holding portion 142 through the pair of openings 142I. When the ultrasound transducer 5 is connected to the holding-case main body 61, the US active-electrode terminal 155 (pair of the blade spring portions 155B) is electrically connected to the US active-electrode terminal 524 by abutting on the US active-electrode terminal 524 in the ultrasound transducer 5.

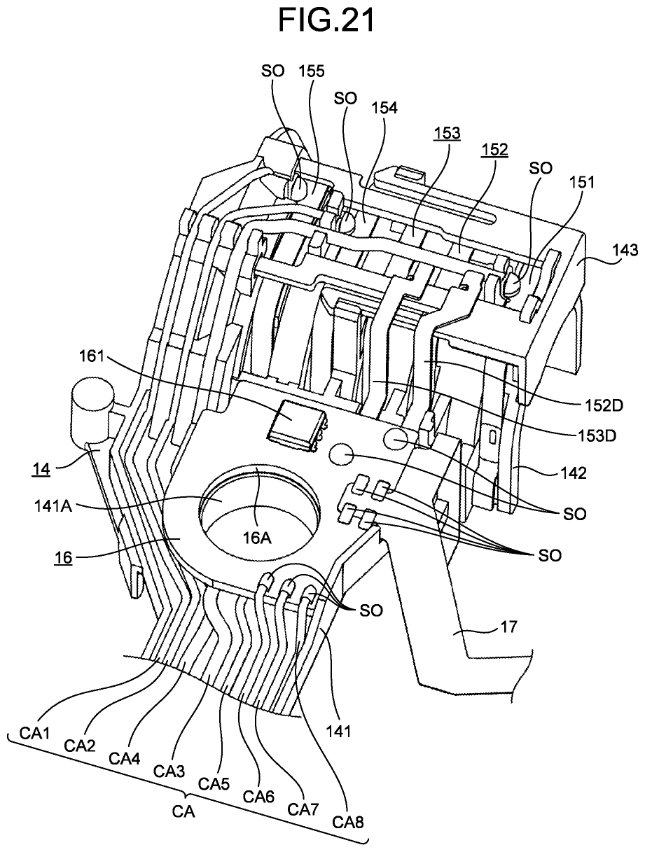

The cable CA is constituted of eight cables of an US active-electrode cable CA1, a US return-electrode cable CA2, an HF return-electrode cable CA3, an HF active-electrode cable CA4, a memory cable CA5, and cables CA6 to CA8 for the first to the third switches (refer to FIG. 21).

The US active-electrode cable CA1 and the US return-electrode cable CA2 form an electrical pathway of the driving signal provided by the control device 3 through the electrical cable CO. The US active-electrode cable CA1 is electrically connected directly with the US active-electrode terminal 155 (refer to FIG. 21).

The HF return-electrode cable CA3 and the HF active-electrode cable CA4 form an electrical pathway of the high frequency signal provided by the control device 3 through the electrical cable CO.

The memory cable CA5 is an electrical pathway that is used for communication between the control device 3 and the TD memory (not illustrated) mounted in the ultrasound transducer 5, and the handpiece memory 161 (refer to FIG. 12) mounted in the circuit board 16.

The first to the third switch cables CA6 to CA8 are cables that electrically connect the electric cable CO with the switch devices SW1 to SW3, respectively.

The US return-electrode terminal 154 corresponds to a second and a third ultrasound terminals. This US return-electrode terminal 154 includes a terminal base portion 154A and a pair of blade spring portions 154B, and has a substantially U-shape as a whole as illustrated in FIG. 10 or FIG. 11.

The terminal base portion 154A has a plate shape in which a length in a longitudinal direction is shorter than the terminal base portion 155A corresponding to an outer diameter size of the step 142C. The terminal base portion 154A is fixed to an outer surface of the step 142C on the
Y axis side in such an orientation that respective plate surfaces are perpendicular to the Y axis.

The pair of blade spring portions 154B are portions respectively extending toward the +Y axis side from both ends of the terminal base portion 154A, and are formed be elastic to be deformable toward the Z axis direction pivoted on the respective ends. These pair of blade spring portions 154B respectively have the same shape as the blade spring portion 155B. Moreover, in the state in which the terminal base portion 154A is fixed to the step 142C, a part of each of the pair of blade spring portions 154B exposes inside the second terminal-holding portion 142 through the pair of openings 142H. When the ultrasound transducer 5 is connected to the holding-case main body 61, the US active-electrode terminal 154 (pair of the blade spring portions 154B) is electrically connected to the US return-electrode terminal 523 by abutting on the US return-electrode terminal 523 in the ultrasound transducer 5.

The US return-electrode cable CA2 is electrically connected directly to the US return-electrode terminal 154 (refer to FIG. 21).

The IR terminal 153 includes a terminal base portion 153A and a pair of blade spring portions 153B, and includes an IR-terminal main body 153C (FIG. 11) having a substantially U-shape as a whole, and a jut-out portion 153D (FIG. 11) that is formed integrally with the IR-terminal main body 153C, and that juts out to the −Z axis side from the terminal base portion 153A.

The terminal base portion 153A has a plate shape in which a length in a longitudinal direction is shorter than the terminal base portion 154A corresponding to an outer diameter size of the step 142B. The terminal base portion 153A is fixed to an outer surface of the step 142B on the −Y axis side in such an orientation that respective plate surfaces are perpendicular to the Y axis.

The pair of blade spring portions 153B are portions respectively extending toward the +Y axis side from both ends of the terminal base portion 153A, and are formed be elastic to be deformable toward the Z axis direction pivoted on the respective ends. These pair of blade spring portions 153B respectively have the same shape as the blade spring portion 155B. Moreover, in the state in which the terminal base portion 153A is fixed to the step 142B, a part of each of the pair of blade spring portions 153B exposes inside the second terminal-holding portion 142 through the pair of openings 142G. When the ultrasound transducer 5 is connected to the holding-case main body 61, the IR terminal 153 (pair of the blade spring portions 153B) is electrically connected to the IR terminal 522 by abutting on the IR terminal 522 in the ultrasound transducer 5.

The HF return-electrode terminal 152 corresponds to a second high-frequency terminal. This HF return-electrode terminal 152 includes a terminal base portion 152A and a pair of blade spring portions 152B, and an HF-return-electrode-terminal main body 152C (FIG. 11) that has a substantially U-shape as a whole, and a jut-out portion 152D (FIG. 11) that is formed integrally with the HF-return-electrode-terminal main body 152C, and that juts out to the −Z axis side from the terminal base portion 152A.

The terminal base portion 152A has a plate shape in which a length in a longitudinal direction is shorter than the terminal base portion 153A corresponding to an outer diameter size of the step 142A. The terminal base portion 152A is fixed to a portion on the proximal end side Ar2 on an outer surface of the step 142C on the −Y axis side in such an orientation that respective plate surfaces are perpendicular to the Y axis.

The pair of blade spring portions 152B are portions respectively extending toward the +Y axis side from both ends of the terminal base portion 152A, and are formed be elastic to be deformable toward the Z axis direction pivoted on the respective ends. These one pair of the blade spring portions 152B have the same shape as the blade spring portion 155B. Moreover, in the state in which the terminal base portion 152A is fixed to the step 142A, a part of each of the pair of blade spring portions 152B exposes inside the second terminal-holding portion 142 through the pair of openings 142F. When the ultrasound transducer 5 is connected to the holding-case main body 61, the HF return-electrode terminal 152 (pair of the blade spring portions 152B) is electrically connected to the HF return-electrode terminal 521 by abutting on the HF return-electrode terminal 521 in the ultrasound transducer 5.

The HF active-electrode terminal 151 includes a terminal base portion 151A and a pair of blade spring portions 151B, and has a substantially U-shape as a whole as illustrated in FIG. 10 or FIG. 11.

The terminal base portion 151A has the same shape as the terminal base portion 152A. The terminal base portion 151A is fixed to a portion on the distal end side Ar1 on an outer surface of the step 142A on the −Y axis side in such an orientation that respective plate surfaces are perpendicular to the Y axis.

The pair of blade spring portions 151B are portions respectively extending toward the +Y axis side from both ends of the terminal base portion 151A, and are formed be elastic to be deformable toward the Z axis direction pivoted on the respective ends. These pair of blade spring portions 151B have the same shape as the blade spring portion 155B. Moreover, in the state in which the terminal base portion 151A is fixed to the step 142A, a part of each of the pair of blade spring portions 151B exposes inside the second terminal-holding portion 142 through the pair of openings 142E. The HF active-electrode terminal 151 (pair of the blade spring portions 151B) is electrically connected to the HF active-electrode terminal 103A by abutting on the HF active-electrode terminal 103A arranged in the probe holder 103.

The HF active-electrode cable CA4 is electrically connected directly to the HF active-electrode terminal 151 (refer to FIG. 21).

As explained above, the respective blade spring portions 151B, 152B, 153B, 154B, 155B in the respective terminals 151 to 155 all have the same shape. Therefore, the contact pressure of the respective terminals 151 to 155 with respect to the respective terminals 103A, 521 to 524 can be set uniform.

FIG. 12 is a diagram illustrating the circuit board 16. Specifically, FIG. 12 is a diagram illustrating an arrangement position of the circuit board 16 in the base unit 13 viewed from the −Y axis side.

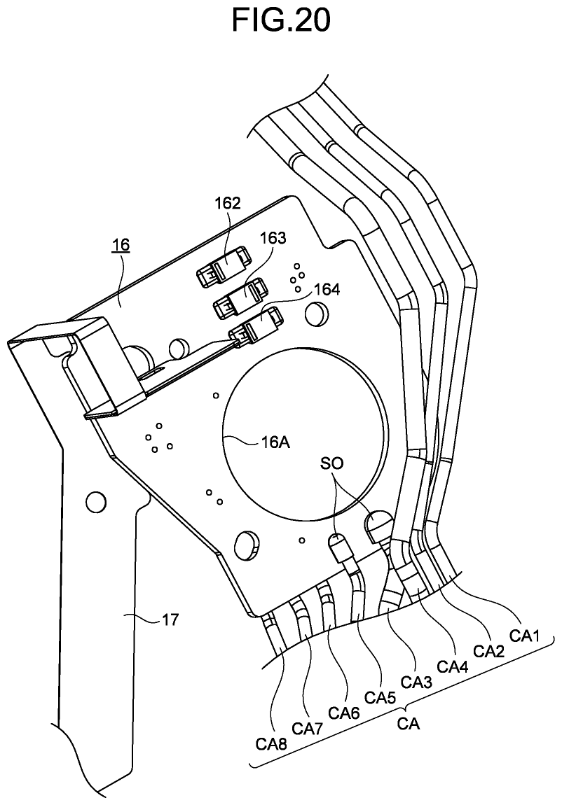

The circuit board 16 is arranged at a position opposing to the bearing hole 141A on a plate surface on the −Y axis side of the base-member main body 141 as illustrated in FIG. 12. In this circuit board 16, a through hole 16A that pierces through to a front side and a rear side, and that communicates with the bearing hole 141A is formed. Moreover, in the circuit board 16, plural electric wirings including a first to a third electric wirings SL1 to SL3 (refer to FIG. 13), the handpiece memory 161 (FIG. 12), and a first to a third diodes 162 to 164 (refer to FIG. 20) corresponding to electric parts are mounted.

The first electric wiring SL1 is electrically connected respectively to the first to the third switch devices SW1 to SW3 through a second electric wiring SL1′ mounted on the flexible board 17 (refer to FIG. 13).

The second electric wiring SL2 is electrically connected respectively to the first and the second diodes 162, 163, and is electrically connected respectively to the first and the second switch devices SW1, SW2 (refer to FIG. 13) through a second electric wiring SL2′ mounted on the flexible board 17.

The third electric wiring SL3 is electrically connected to the third diode 164, and is electrically connected to the third switch SW3 through a third electric wiring SL3′ mounted on the flexible board 17 (refer to FIG. 13).

To the circuit board 16, the first to the third switch cables CA6 to CA8 are respectively connected. Thus, the first to the third electric wiring SL1 to SL3 are electrically connected to the first to the third switch cables CA6 to CA8, respectively.

The handpiece memory 161 corresponds to a memory. This handpiece memory 161 stores identification information to identify, for example, the handpiece 4. To the circuit board 16, the jut-out portion 153D in the IR terminal 153, the jut-out portion 152D in the HF return-electrode terminal 152, the memory cable CA5, and the HF return-electrode cable CA3 are respectively connected. Thus, the handpiece memory 161 is electrically connected respectively to the memory cable CA5 that functions as a data line used for communication with the control device 3, and to the HF return-electrode cable CA3 that functions as a ground line used for communication with the control device 3 through a pair of electric wiring (not illustrated) mounted on the circuit board 16. Moreover, the handpiece memory 161 is electrically connected respectively to the IR terminal 153 and the HF return-electrode terminal 152 through the pair of electric wirings. That is, the TD memory (not illustrated) mounted in the ultrasound transducer 5 is electrically connected respectively to the memory cable CA5 and the HF return-electrode cable CA3, similarly to the handpiece memory 161.

The flexible board 17 is connected to the circuit board 16, and extends respectively to an arrangement position of the first and the second switches 8A, 8B, and to an arrangement position of the metal contact 19 (refer to FIG. 16, FIG. 17) attached to the switch supporting portion 18 from a position at which the flexible board 17 is connected to the circuit board 16. To this flexible board 17, the first to the third electric wirings SL1′ to SL3′, and the first and the second switch devices SW1, SW2 are mounted.

The first electric wiring SL1′ is a wiring that relays the first electric wiring SL1 to the first to the third switch devices SW1 to SW3 (refer to FIG. 13).

The second electric wiring SL2′ is a wiring that relays the second electric wiring SL2 to the first and the second switch devices SW1, SW2 (refer to FIG. 13).

The third electric wiring SL3′ is a wiring that relays the second electric wiring SL2 to the third switch device SW3 (refer to FIG. 13).

A part of the first electric wiring SL1′ and a part of the third electric wiring SL3′ expose outside the flexible board 17 at a position opposing to the metal contact 19 (FIG. 10). The part of the first electric wiring SL1′ and the part of the third electric wiring SL3′, and the metal contact 19 constitute the third switch device SW3.

The first switch device SW1 is arranged at a position opposing to the first switch 8A (FIG. 5), and detects a setting operation of the first energy output mode made with respect to the first switch 8A.

The second switch device SW2 is arranged at a position opposing to the second switch 8B, and detects a setting cooperation made with respect to the second switch 8B (FIG. 5).

FIG. 13 is a circuit diagram for detecting an operation made with respect to the first to the third switches 8A to 8C.

The control device 3 recognizes that an operation has been made with respect to the first to the third switches 8A to 8C as described below.

When a setting operation of the first energy output mode is made with respect to the first switch 8A, the first and the second electric wirings SL1′, SL2′ become continuous by the first switch device SW1. By the first to the third diodes 162 to 164, an electric current flows only to the first switch cable CA6 (the first electric wirings SL1, SL1′) from the second switch cable CA7 (the second electric wirings SL2, SL2′). By recognizing the flow of the electric current, the control device 3 recognizes that a setting operation of the first energy output mode has been made with respect to the first switch 8A.

When a setting operation of the second energy output mode is made with respect to the second switch 8B, the first and the second electric wirings SL1′, SL2′ become continuous by the second switch SW2. By the first to the third diodes 162 to 164, an electric current flows only to the second switch cable CA7 (the second electric wirings SL2, SL2′) from the first switch cable CA6 (the first electric wirings SL1, SL1′). By recognizing the flow of the electric current, the control device 3 recognizes that a setting operation of the second energy output mode has been made with respect to the second switch 8B.

When a change operation is made with respect to the third switch 8C, the first and the third electric wirings SL1′, SL3′ become a continuous state or a discontinuous state by the third switch device SW3. In the continuous state, by the first to the third diodes 162 to 164, an electric current flows only to the first switch cable CA6 (the first electric wirings SL1, SL1′) from the third switch cable CA8 (the third electric wirings SL3, SL3′). By recognizing the flow of the electric current, the control device 3 recognizes whether a change operation has been made with respect to the third switch 8C.

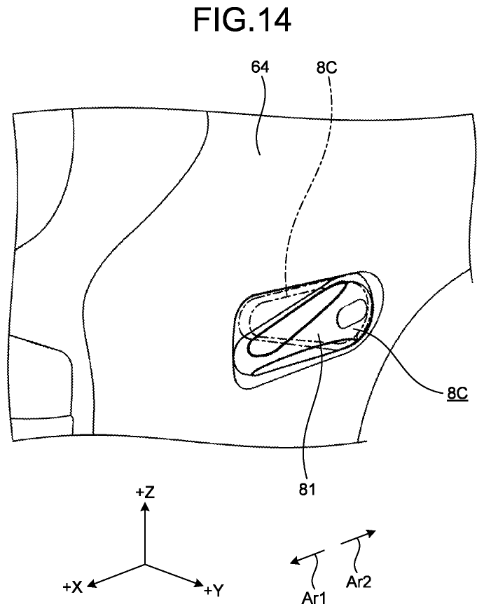

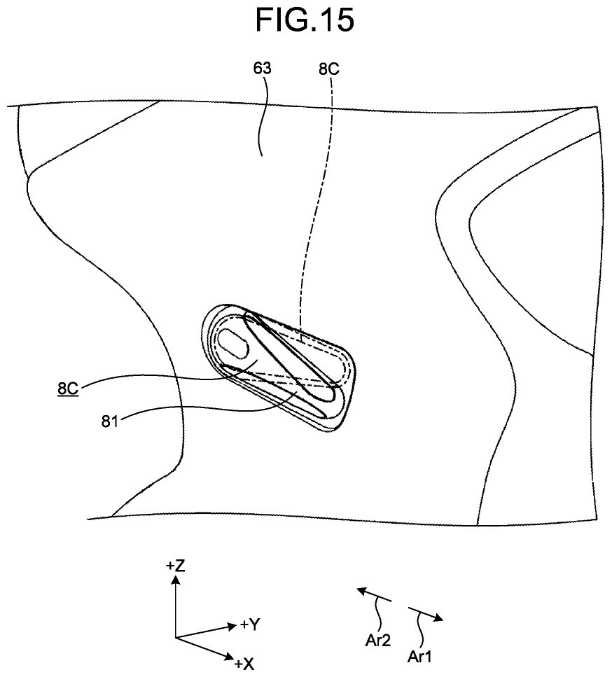

FIG. 14 to FIG. 17 are diagrams for explaining a supporting structure of the third switch 8C. Specifically, FIG. 14 illustrates the holding case 6 viewed from the +Y axis side. FIG. 15 illustrates the holding case 6 viewed from the −Y axis side. FIG. 16 and FIG. 17 illustrate the switch supporting portion 18 viewed from the +Y axis side.

Before explaining a structure of the switch supporting portion 18, a structure of the third switch 8C will be explained.

The pair of third switches 8C have the same shape. The third switch 8C includes a tub portion 81 and an axis portion 82 as illustrated in FIG. 5, or FIG. 14 to FIG. 17.

The tub portion 81 is a portion accepting a change operation by an operator such as a surgeon. In the present embodiment, the tub portion 81 has a tapered shape gradually becoming thin as it approaches the distal end side Ar1.

The axis portion 82 protrudes along the Y axis from the proximal end side Ar2 of the tub portion 81. In the present embodiment, the axis portion 82 has a rectangular cross-sectional shape. Out of the pair of third switches 8C, the axis portion 82 of the third switch 8C on the +Y axis side is inserted in a circular hole 641 (FIG. 5) that pierces through to a front side and a rear side of the second housing 64, and protrudes to the inside of the holding case 6. On the other hand, the axis portion 82 of the third switch 8C on the −Y axis side is inserted in a circular hole (not illustrated) that pierces through to a front side and a rear side of the first housing 63, and protrudes to the inside of the holding case 6.

The switch supporting portion 18 is made from a material having an electrical insulation property, and is arranged at a position opposing to the bearing hole 141A on a plate surface of the base-member main body 141 on the +Y axis side as illustrated in FIG. 5, FIG. 16, or FIG. 17. This switch supporting portion 18 includes a supporting-portion main body 181 and a spring portion 182.

The supporting-portion main body 181 extends along the Y axis, as illustrated in FIG. 5, and includes a columnar axis 181A in a cylindrical shape that is inserted in the bearing hole 141A. An outer diameter size of the columnar axis 181A is set to be a little larger than an inner diameter size of the bearing hole 141A. The switch supporting portion 18 is structured rotatable about the third rotation axis Rx3, having the columnar axis 181A axially supported by the bearing hole 141A.

Moreover, in the columnar axis 181A, fitting holes 81B (FIG. 5, FIG. 16, FIG. 17) having a rectangular cross-sectional shape in which the respective axis portions 82 of the pair of third switches 8C piercing through along the Y axis and protruding to the inside of the holding case 6 fit are formed. That is, the pair of third switches 8C are axially supported in a rotatable manner about the third rotation axis Rx3 at a substantially central position in the Y axis direction inside the holding case 6 by the bearing hole 141A and the columnar axis 181A.

The spring portion 182 is a portion that protrudes to the proximal end side Ar2 from an end portion on the −Z axis side of the supporting-portion main body 181, and that extends to the +Z axis side in a bending manner, and is formed be elastic to be deformable toward the X axis direction pivoted on the end portion on the −Z axis side of the supporting-portion main body 181. Moreover, in the spring portion 182, a protrusion 182A that protrudes toward the proximal end side Ar2 is arranged at an end portion on the +Z axis side.

In the base-member main body 141, an engaging protrusion 144 that protrudes to the +Y axis side from a position on the proximal end side Ar2 relative to the switch supporting portion 18 is formed on a plate surface on the +Y axis side as illustrated in FIG. 16 or FIG. 17. Moreover, in the engaging protrusion 144, a pair of engaging concave portions 144A, 144B corresponding to a shape of the protrusion 182A in the spring portion 182 are aligned in the Z axis direction on a side surface on the distal end side Ar1.

The metal contact 19 is attached to an end portion on the +Z axis side of the switch supporting portion 18 as illustrated in FIG. 16 or FIG. 17. The metal contact 19 constitutes the third switch device SW3. That is, the metal contact 19 makes the first and the third electric wiring SL1′, SL3′ continuous by abutting on a part of the first and the third electric wirings SL1′, SL3′ (FIG. 10) exposed to the outside of the flexible board 17, respectively. Moreover, the metal contact 19 brings the first and the third electric wirings SL1′, SL3′ to a discontinuous state by separating from the part of the first and the third electric wirings SL1′, SL3′.

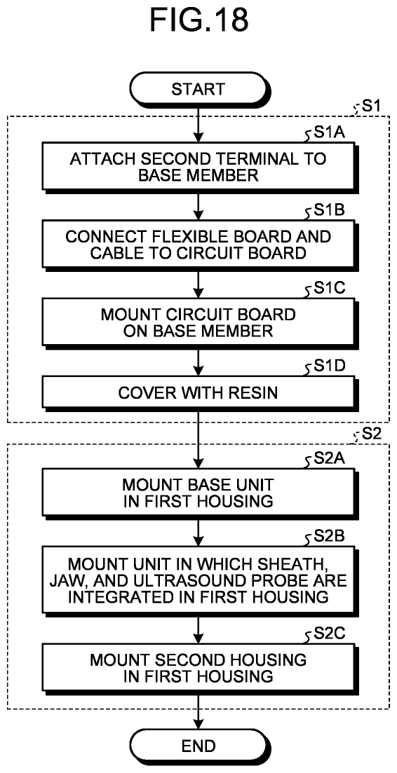

When a portion of the third switch 8C on the distal end side Ar1 is brought down to the −Z axis side (refer to the third switch 8C indicated by a solid line in FIG. 14, FIG. 15), the switch supporting portion 18 rotates in a counterclockwise direction in FIG. 18 about the third rotation axis Rx3, to be a state illustrated in FIG. 16. At this time, the metal contact 19 is separated from the part of the first and the third electric wirings SL1′, SL3′ exposed to the outside of the flexible board 17. That is, the first and the third electric wirings SL1′, SL3′ become the discontinuous state.

On the other hand, when the portion of the third switch 8C on the distal end side Ar1 is brought down to the +Z axis side (refer to the third switch 8C indicated by an alternate long and short dashed lines in FIG. 14, FIG. 15), the switch supporting portion 18 rotates in a clockwise direction in FIG. 17 about the third rotation axis Rx3, to be a state illustrated in FIG. 17. At this time, the metal contact 19 abuts on the part of the first and the third electric wirings SL1′, SL3′ exposed to the outside of the flexible board 17. That is, the first and the third electric wirings SL1′, SL3′ become continuous.

When both the portion of the third switch 8C on the distal end side Ar1 is brought down to the −Z axis side and the portion of the third switch 8C on the distal end side Ar1 is brought down to the +Z axis side, the spring portion 182 slides on a side surface on the distal end side Ar1 of the engaging protrusion 144 while making an elastic deformation in the X axis direction. The protrusion 182A then engages with the engaging concave portion 144A positioned on the +Z axis side when the portion of the third switch 8C on the distal end side Ar1 is brought down to the −Z axis side (FIG. 16). Moreover, the protrusion 182A engages with the engaging concave portion 144B positioned on the −Z axis side when the portion of the third switch 8C on the distal end side Ar1 is brought down to the +Z axis side (FIG. 17). According to the engagement of the protrusion 182A with the engaging concave portions 144A, 144B, vibrations are generated in the pair of third switches 8C. That is, a click feeling is given to an operator that operates the pair of third switches 8C. By thus structuring, the third switch 8C can prevent switching by mistake even when it is touched unintendedly by a finger or the like of the operator, and enables to perform a switching operation easily without applying an excessive force. Furthermore, because the pair of third switches 8C operate in a linked manner, it can be operated by either a right-hander or a left-hander, and it is possible to check which mode is on by visually recognizing a position of the portion on the distal end side Ar1.

Manufacturing Method of Energy Treatment Instrument Next, a manufacturing method of the energy treatment instrument 2 described above will be explained.

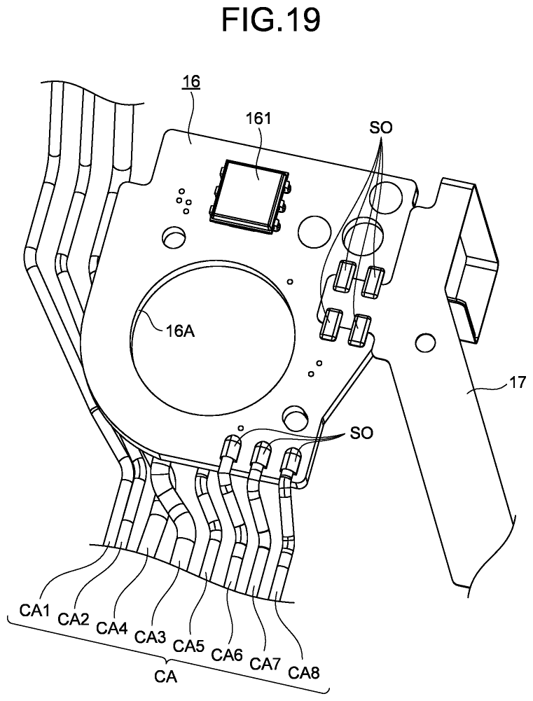

FIG. 18 is a flowchart illustrating the manufacturing method of the energy treatment instrument 2. FIG. 19 to FIG. 22 are diagrams for explaining the manufacturing method of the energy treatment instrument 2.

Steps S1, S2 described below are performed in different sites. Specifically, step S2 is performed in a site such as a clean room in which cleanness (cleanliness) is relatively high (hereinafter, denoted as second site). On the other hand, step S1 is performed in a site, such as a clean room in which cleanness (cleanliness) is lower than the second site (hereinafter, denoted as first site).

In the following, steps S1, S2 are sequentially explained.

About Step S1

At step S1, a worker assembles the base unit 13 as described below in the first site.

The worker attaches the second terminal 15 to the base member 14 (step S1A).

After step S1A, the worker connects the flexible board 17 and the respective cables CA3, CA5 to CA8 to the circuit board 16 by solder SO as illustrated in FIG. 19 and FIG. 20 (step S1B).

After step S1B, the worker sets the circuit board 16 as described below with respect to the base member 14 (step S1C).

Specifically, the worker fixes the cable CA to the base member 14 with the tie band CT. Moreover, the worker connects the respective cables CA4, CA2, CA1 to the respective terminals 151, 154, 155 by the solder SO as illustrated in FIG. 21. Furthermore, the worker connects the respective protruding portions 152D, 153D of the respective terminals 152, 153 by the solder SO to the circuit board 16.

After step S1C, the worker covers a plate surface on which the circuit board 16 is arranged in the base-member main body 141 with a resin RE, such as epoxy resin, as illustrated in FIG. 22 (step S1D).

About Step S2 At step S2, the worker assembles the energy treatment instrument 2 as described below in the second site.

The worker mounts the base unit 13 assembled at step S1 in the first housing 63 from the +Y axis side (step S2A).

After step S2A, the worker mounts a unit in which the rotating knob 9, the sheath 10, the jaw 11, and the ultrasound probe 12 are integrated in the first housing 63 from the +Y axis side step S2B). At this time, the end portion of the ultrasound probe 12 on the proximal end side Ar2 is arranged inside the second terminal holding portion 142 through the notch 142J formed in the second terminal holding portion 142.

After step S2B, the worker mounts the second housing 64 in the first housing 63 (step S2C) and, moreover, mounts the pair of third switches 8C in the first and the second housings 63, 64, respectively.

As described above, at steps S2A to S2C, the unit in which the base unit 13, the rotating knob 9, the sheath 10, the jaw 11, and the ultrasound probe 12 are integrated, and the second housing 64 are assembled from all from the same direction (from the +Y axis side).

By above steps S1, S2, the energy treatment instrument 2 is manufactured.

According to the present embodiment explained above, following effects are produced.

The energy treatment instrument 2 according to the present embodiment includes the base unit 13 described above. Therefore, when assembling the energy treatment instrument 2 in the second site, the energy treatment instrument 2 can be assembled by performing steps S2A to S2C, and a wiring work of the electric cable CO and the ultrasound transducer 5 and the like is not necessary to be performed.

Therefore, according to the energy treatment instrument 2 according to the present embodiment, the number of processes of assembling the energy treatment instrument 2 in the second site can be reduced, and the assemblability can be improved.

Moreover, in the energy treatment instrument 2 according to the present embodiment, the second terminal 15 is structured such that the HF active-electrode terminal 151, the HF return-electrode terminal 152, the IR terminal 153, the US return-electrode terminal 154, and the US active-electrode terminal 155 are aligned along the center axis Ax sequentially from the distal end side Ar1.

That is, the HF return-electrode terminal 152 and the US return-electrode terminal 154 are set to have electric potentials identical or close to each other, and are arranged in the center of the five terminals 151 to 155. In other words, even if the HF return-electrode terminal 152 and the US return-electrode terminal 154 become short-circuited, an influence on the functions or the like is small. The HF return-electrode terminal 152 and HF active-electrode terminal 151; and the US active-electrode terminal 155 and the US return-electrode terminal 154 respectively have a potential difference to supply an electric energy. Therefore, unlike the HF return-electrode terminal 152 and the US return-electrode terminal 154, the HF active-electrode terminal 151 and the US active-electrode terminal 155 can affect the functions when a short circuit occurs. By aligning the respective terminals 151 to 155 in the arrangement as this example, it is possible to prevent the HF active-electrode terminal 151 and the US active-electrode terminal 155 from becoming short-circuited and, therefore, the reliability of the energy treatment instrument 2 can be ensured.

Moreover, in the energy treatment instrument 2 according to the present embodiment, the base unit 13 includes the circuit board 16 that relays the HF return-electrode terminal 152, the IR terminal 153, and the cable CA. Therefore, by mounting the handpiece memory 161 and the first to the third diodes 162 to 164 on the circuit board 16, various kinds of functions can be provided to the circuit board 16.

Particularly, by mounting the first to the third diodes 162 to 164 on the circuit board 16, an operation made with respect to the first to the third switches 8A to 8C can be detected by using three wirings of the first to the third electric wirings SL1 to SL3. Therefore, only three wirings of the first to the third switch cables CA6 to CA8 are necessary as the switch cables constituting the cable CA, and the cable CA can be made thin.

Moreover, in the energy treatment instrument 2 according to the present embodiment, the base unit 13 includes the flexible board 17 that relays the first to the third switch devices SW1 to SW3 and the circuit board 16. Accordingly, when assembling the energy treatment instrument 2 in the second site, a wiring work of the electric cable CO and the first to the third switch devices SW1 to SW3 is not necessary to be performed. Therefore, the assemblability of the energy treatment instrument 2 can be further improved.

Furthermore, in the energy treatment instrument 2 according to the present embodiment, at step S1D, the plate surface on which the circuit board 16 is arranged in the base-member main body 141 is covered with the resin RE. Therefore, a most part of the respective cables CA1 to CA6 is sealed with the resin RE, and the electrical insulation can be sufficiently obtained.

### Other Embodiments

An embodiment to implement the present embodiment has so far been explained, but the disclosure is not limited to the embodiment descried above.

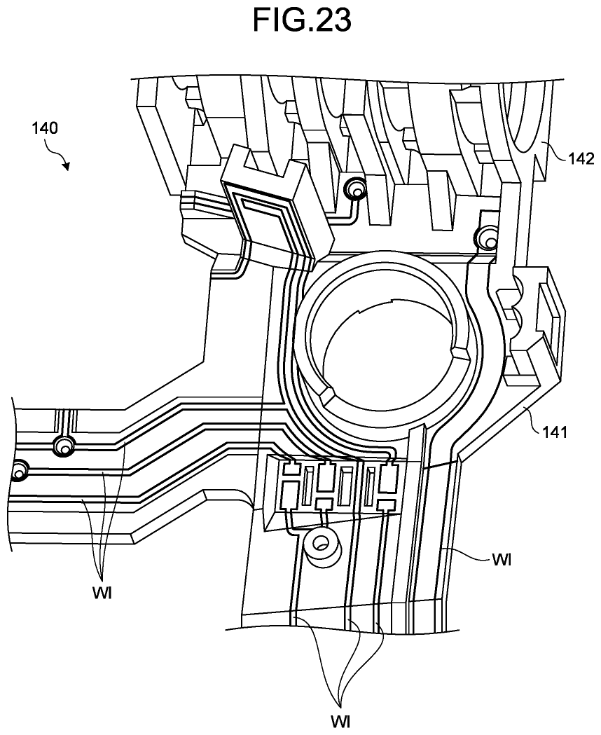

FIG. 23 is a diagram illustrating a modification of the present embodiment.

In the embodiment described above, a base member 140 illustrated in FIG. 23 may be used in place of the base member 14.

The base member 140 according to the present modification is constituted of a molded interconnect device (MID). That is, the base member 140 is constituted of a resin mold in which a wiring WI is formed on an outer surface as illustrated in FIG. 23.

In the embodiment described above, a configuration in which both an ultrasound energy and a high frequency energy are applied to a target area is applied as the energy treatment instrument according to the disclosure, but it is not limited thereto. A configuration in which at least one of an ultrasound energy, a high frequency energy, and a heat energy is applied may be applied. “Application of a heat energy to a target area” signifies transmission of heat generated by a heater or the like to a target area.

According to the energy treatment instrument of the disclosure, assemblability can be improved.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An energy treatment instrument comprising:
a grip configured to be gripped by an operator;
a transducer configured to generate ultrasound vibrations according to a supplied electric power;
a first ultrasound terminal that is arranged in the transducer;
a first high-frequency terminal that is arranged in the grip; and
a base unit that is arranged in the grip, wherein
the base unit includes:
a second ultrasound terminal abutting the first ultrasound terminal in order to electrically connect the first ultrasound terminal and a cable electrically connected to a control device outside the energy treatment instrument; and
a second high-frequency terminal abutting the first high-frequency terminal in order to electrically connect the first high-frequency terminal and a cable electrically connected to the control device outside the energy treatment instrument.

2. The energy treatment instrument according to claim 1, the ultrasound transducer including:
a plurality of piezoelectric devices configured to generate ultrasound vibrations based on an alternating current;
a front mass;
a back mass that is electrically connected to an end effector, the back mass being configured to sandwich the piezoelectric devices that are laminated, with the front mass; and
a first electrode plate and a second electrode plate that are arranged alternately between adjacent piezoelectric devices, the first electrode plate and the second electrode plate being supplied with the alternating current, wherein:
the first high-frequency terminal is electrically connected to the back mass,
a pair of second ultrasound terminals that include:
a third ultrasound terminal electrically connected to the first electrode plate arranged at a position closer to the back mass than to the first electrode plate and the second electrode plate; and
a fourth ultrasound terminal that is electrically connected to the second electrode plate through another one of the pair of first electrode terminals, and
the third ultrasound terminal is arranged between the fourth ultrasound terminal and the second high-frequency terminal.

3. An energy treatment instrument comprising:
an end effector configured to apply an energy to treat a living tissue;
a grip configured to support the end effector;
a first terminal that is electrically connected to the end effector, the first terminal and the end effector being configured to rotate with respect to the grip about a center axis of the end effector;
a switch device configured to execute an operation; and
a base unit that is arranged in the grip, the base unit including:
a second terminal abutting the first terminal in order to electrically connect the first terminal and a cable electrically connected to a control device outside the energy treatment instrument;
a circuit board configured to relay between the second terminal and the cable; and
a flexible board configured to relay between the switch device and the circuit board.

4. The energy treatment instrument according to claim 3, further comprising:
three switch devices each configured to execute an operation, wherein:
on the circuit board, three electric wirings and an electronic part are mounted on the circuit board,
the three electric wirings are configured to electrically connect the three switch devices, respectively, and
the electronic part is configured to detect the operation executed by each of the three switch devices by using the three electric wirings are mounted.

5. The energy treatment instrument according to claim 3, wherein
on the circuit board, a memory configured to store information is mounted.

6. The energy treatment instrument according to claim 3, wherein
at least a part of a surface of the base unit is covered with resin.

7. The energy treatment instrument according to claim 3, wherein
the cable is attached to the base unit.

8. An energy treatment instrument comprising:
a transducer that includes a first terminal, the transducer being configured to generate ultrasound vibrations by an electric power supplied from the first terminal;
a grip that is gripped by an operator; and
a base unit that is arranged in the grip, the base unit including:
a second terminal configured to abut on the first terminal;
a cable configured to electrically connect the second terminal and a control device spaced apart from and connected to the transducer, the grip and the base unit; and
a base member including a fixing portion that is connected to the second terminal and the cable, the fixing portion being configured to fix the second terminal and the cable in the grip.

9. The energy treatment instrument according to claim 8, wherein
the base member includes a hole to be fixed in the grip with screws.

10. The energy treatment instrument according to claim 8, wherein
the cable comprises a plurality of cables, and the base member includes a band to put the cables together.

11. The energy treatment instrument according to claim 8, wherein
the base member includes a second-terminal holding member having an inner diameter larger than an outer diameter of the transducer, and the second terminal is arranged outside the second-terminal holding member.
